(19) United States
(12) Patent Application Publication
(10) Pub. No.: US 2019/0205839 A1
(43) Pub. Date: Jul. 4, 2019

(54) ENHANCED COMPUTER EXPERIENCE FROM PERSONAL ACTIVITY PATTERN

(71) Applicant: MICROSOFT TECHNOLOGY LICENSING, LLC, Redmond, WA (US)

(72) Inventors: Dikla Dotan-Cohen, Herzliya (IL); Ido Priness, Herzliya (IL); Haim Somech, Herzliya (IL)

(21) Appl. No.: 15/858,966

(22) Filed: Dec. 29, 2017

(51) Int. Cl. G06Q 10/10; H04L 29/08; A61B 5/11

(52) U.S. Cl. CPC G06Q 10/1093; A61B 5/1118; H04L 67/22

(57) ABSTRACT

An enhanced computing experience is provided to a user based on an optimal schedule of user activity or activity types. The optimal schedule may be generated based on learned user activity patterns and determined or inferred future activity likely to be performed by the user. A user's current context and user preferences also may be considered when generating an optimal schedule. The optimal schedule may be utilized by a computerized personal assistant application to provide an enhanced computing experience for the user. For example, a calendar management program manages a user's calendar events to be more in accordance with an optimal schedule; a personal performance application generates a recommended daily schedule for the user; and a notifications service manages electronic notifications to the user or to other people in accordance with an optimal schedule.

### BACKGROUND

The use of computing devices has positively impacted human productivity but has also increased reliance on these devices for information, recommendations, and other services that assist them in scheduling and managing day-to-day tasks, meetings, and other activities. For example, users of these computing devices increasingly rely on computer calendar applications or scheduling applications for managing their time.

Users of these computing devices, and particularly workers, often have repeating patterns of activity. For example, a user may open a word document each morning on working days and perform focus-intensive work during this time that is best facilitated with minimal disruption, or a user may eat lunch or travel to pick up family members at common times on working days. Moreover, user working patterns and work preferences are often personal to a user and may be different from other users. For instance, some users may prefer to each lunch out of the office, while others eat at the office or even tend to snack on something between meetings. Some users may prefer holding back-to-back meetings so that longer slots of time may be left available for quiet focus work, while other users become overwhelmed when too many meetings are scheduled close together. Some users prefer to use their commute time for online meetings, while other users wouldn't even consider this as an option. However, conventional computer calendar and scheduling applications tend to provide a one-size-fits-all approach to scheduling or managing user time. Consequently, instead of being helpful, these computer-performed calendar and scheduling services may contribute to user inefficiency, fatigue, and poor workflow management. For example, these computerized scheduling programs are generally incapable of learning user behavior patterns or adapting to changes in user behavior, and incorporating this information into the scheduling services they provide. Accordingly, determining optimal activity schedules for a user that best utilize the user's time, availability, energy, workload capacity, and other resources based on user activity information that can be sensed or determined by the computing device remains a technical challenge.

### SUMMARY

This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

In brief, and at a high level, this disclosure describes, among other things, methods and systems, which may be embodied in one or more computer applications or services, that may provide a user with an enhanced computing experience by determining and utilizing an optimal user-activity schedule. In particular, embodiments may determine one or more optimal activity schedules for a user, based on learned behavior patterns of the user. Data corresponding to user behavior (or user activity) may be gathered over time, such as from sensors of one or more of the user's computing devices. From this historical user activity information, a computer system may determine a user's activity patterns. Future activity likely to be performed by the user also may be determined or inferred and used by the computing system in conjunction with the learned activity patterns to generate one or more optimal activity schedules. A generated schedule may be considered optimal for the user because it is consistent with learned behavior patterns of the user. For example, where a user has a pattern of doing focused work at certain periods of the day, a generated optimal schedule may allocate focus-intensive work during those periods or may avoid scheduling meetings during those times. In some embodiments, a user's current context also may be used for determining or selecting optimal schedules. Additionally, in some embodiments, user preferences or settings may be considered when generating optimal schedules.

The optimal schedule(s) then may be utilized by one or more computerized personal assistant applications or services described herein to provide an enhanced computing experience for the user. For example, a calendar management program may utilize an optimal schedule for managing a user's calendar events to be more in accordance with the optimal schedule; a personal performance optimizer application may utilize an optimal schedule to provide the user with a recommended daily schedule of user activity based on future activity to be performed by the user; or a notifications service may utilize an optimal schedule to manage electronic notifications to the user or to other people. In this way, these computer applications and services improve activity scheduling and workflow optimization and can provide an enhanced user experience that best utilizes the user's time, availability, energy, and workload capacity. Moreover, by analyzing user activity to determine or infer user activity patterns, an optimal schedule of user activity or activity types may be determined and utilized by computer applications or services, thereby personalizing a computing experience for the user in a way that more effectively supports their activity and working needs.

### BRIEF DESCRIPTION OF THE DRAWINGS

The subject matter provided in this disclosure is discussed with reference to the attached drawing figures, which depict exemplary and non-limiting embodiments of the technology, wherein:

FIG. 1 is a block diagram of an example operating environment suitable for implementing embodiments of the present disclosure;

FIG. 2 is a diagram depicting an example computing architecture suitable for implementing embodiments of the present disclosure;

FIGS. 3A and 3B depict example aspects of optimal schedules generated in accordance with some embodiments of the present disclosure;

FIG. 4 is a block diagram of an example method for generating an optimal user activity schedule and providing an enhanced experience, in accordance with an embodiment of the present disclosure;

FIG. 5 is another block diagram of an example method for generating an optimal user activity schedule and providing an enhanced experience, in accordance with an embodiment of the present disclosure;

FIGS. 6A-6C depict an illustrative example of an enhanced user experience provided by a calendar managing application, in accordance with an embodiment of the present disclosure; and

FIG. 7 is a block diagram of an example computing environment suitable for use in implementing embodiments of the present disclosure.

### DETAILED DESCRIPTION

The subject matter of the present disclosure is described with specificity herein to meet statutory requirements. However, the description itself is not intended to limit the scope of this patent. Rather, the inventors have contemplated that the claimed subject matter might also be embodied in other ways, to include different steps, or combinations of steps, similar to the ones described in this document and in conjunction with other present or future technologies. Moreover, although the terms “step” and “block” may be used herein to connote different elements of methods employed, the terms should not be interpreted as implying any particular order among or between various steps or blocks disclosed herein unless and except when the order is explicitly described and required.

Aspects of the technology described herein are directed towards systems, methods, and computer storage media for, among other things, providing enhanced computing experiences for a user based on optimal schedules of user activity generated from learned patterns of user activity, and in some instances, the user's context and/or preferences. In particular, embodiments may determine one or more optimal activity schedules for a user, based on learned behavior or activity patterns of the user. Data corresponding to user behavior (or user activity) may be gathered over time, such as from sensors associated with one or more of the user's computing devices. From this historical user activity information, a computer system may determine the user's activity patterns. Future activity likely to be performed by the user also may be determined or inferred and used by the computing system in conjunction with the learned activity patterns to generate one or more optimal activity schedules. Examples of future activity may include meetings, calendar events, work-related tasks, reading, writing, traveling or commuting, calling or communicating, responding to or handling emails, eating, working out, shopping, relaxing, listening to or watching media, sleeping, or nearly any other user activity or task that may be detected or inferred by computing device(s).

The term “optimal” as in an optimal schedule, is used broadly herein, and at least in some embodiments, is not necessarily intended to imply only one schedule or an ideal schedule. But rather, as described in some embodiments herein, one or more optimal schedules may be determined and each may be considered as optimized or preferred for a user in some manner, for example and without limitation, according to user behavior or routine, user context, and/or user preferences. Moreover, according to some embodiments further described herein, a particular optimal schedule may be selected or determined from a plurality of optimal schedules, based on user data (and/or data determined from user data), such as a current context of a user, user preferences, or user feedback, activity pattern information, or other user data, for example and without limitation.

The term “activity pattern” is used broadly herein and may refer to a plurality of user interactions conducted using one or more user devices, behavior or activity performed by the user determinable by one or more user devices, events (including actions) related to user activity, or any type of user activity determinable via a computing device, wherein the plurality of interactions, actions, events, or activity share common features or characteristics, thereby forming a pattern. In some embodiments, these in-common features or variables may comprise features characterizing a user activity, time, location, or other contextual information associated with the user activity, as further described herein. Examples of user activity patterns may include, without limitation, activity patterns based on time (e.g., a user often accesses and reads documents at certain times of the day), location (e.g., upon arriving at work in the morning, a user typically spends thirty minutes to one hour responding to emails), or other context, as described herein. In some embodiments, a semantic analysis is performed on the activity events and associated information to characterize aspects of the activity event. The semantic analysis may be used to characterize information associated with the activity event and may provide other relevant features of the activity events that may be used to identify patterns.

As described above, the generated schedule(s) may be considered optimal for the user by being consistent with learned behavior patterns (or activity patterns) of the user. For example, where a user has a pattern of doing focused work at certain periods of the day, a generated optimal schedule may allocate focus-intensive work during those periods or may avoid scheduling meetings during those times. In some embodiments, a user's current context may also be used for determining or selecting optimal schedules. For example, where a current context indicates that a user has an upcoming project deadline, a schedule may be generated (or an already generated schedule may be selected) that includes more time slots allocated for working on the project. In some instances, multiple optimal schedules (or candidate optimal schedules) may be generated which may be mutually exclusive, and a current context may be used to select an appropriate optimal schedule. For example, where context indicates the user is traveling and out of the office, then an optimal schedule for traveling may be selected over an optimal schedule that includes office meetings. Additionally, in some embodiments, user preferences or settings may be considered when generating optimal schedules, thereby enabling a user to tune the automated scheduling assistance services according to their preferences.

In some embodiments, the determined optimal schedule(s) may be made available to one or more computerized personal assistant tools, which may be embodied as one or more computer applications (or apps) or services, which consume the schedule information to provide an enhanced user experience. It should be noted that the term application or app is used broadly herein, and generally refers to a computer program or computer application, which may comprise one or more programs or services, and may run on the user's device(s), or in the cloud. For example, in one embodiment, the optimal schedule information may be provided via an Application Programming Interface (API) so that other computing applications and services, which may include computing applications and services by third parties, can use it. For example and without limitation, one such computer application or service may include a calendar optimization program for scheduling, rescheduling, rearranging, and/or managing events on a user's calendar so that the events are more consistent with the user's determined work patterns. Another example includes a personal performance optimizer service, which, in one embodiment, may run on a user's personal computing device, and which may provide recommendations, such as a daily suggested schedule, or which may automatically facilitate managing a user's time based on the user's determined work patterns and current tasks or obligations of the user. Yet another example includes a notifications computing service that may notify other people regarding times a user is available or prefers not to be disturbed, based on the determined user activity patterns. For instance, where the user activity patterns indicate the user is typically involved in focused work, the notifications service may automatically indicate to other people that the user is unavailable or busy. In one embodiment, the notifications service may throttle or control notifications presented to the user so that they are presented at optimal times consistent with the determined user activity patterns. For instance, where the user patterns indicate the user is typically involved in focused work, notifications may be withheld or may be provided in a format that does not interrupt, such as through a graphic user interface element (e.g., an icon or visual indicator) indicting the notification, rather than an audible notification.

In this way, the computer applications and services described herein improve activity scheduling and workflow optimization and can further provide an enhanced user experience that best utilizes the user's time, availability, energy, and workload capacity. Moreover, embodiments remove subjectivity that would otherwise be present in conventional approaches to scheduling optimization, such as approaches that may be performed by humans or conventional computerized scheduling applications. For example, conventional computerized scheduling services typically merely receive user input for scheduling activity or at most, may recommend scheduling meetings at a first available time for the user or other meeting invitees. Further, when determining a schedule using these conventional services, users often believe (or hope) that they will accomplish more activity within a designated timeframe then they actually will, and thus will not schedule an appropriate amount of time needed for performing the activity. Similarly, users may neglect to schedule time for other activity that they regularly perform. Still further, the designated timeframes for activity are not necessarily optimal to the particular user's routine, workflow, or current context. Consequently, these computerized scheduling services do not account for activity that the user will likely perform and schedule activity at suboptimal times. Further, the recommendations (such as scheduling suggestions), alerts, or notifications that may be provided by these computerized scheduling services are also less accurate, less likely to be adhered to, and/or lead to further inefficiencies due to these underlying problems (all of which are addressed and improved upon by embodiments described herein).

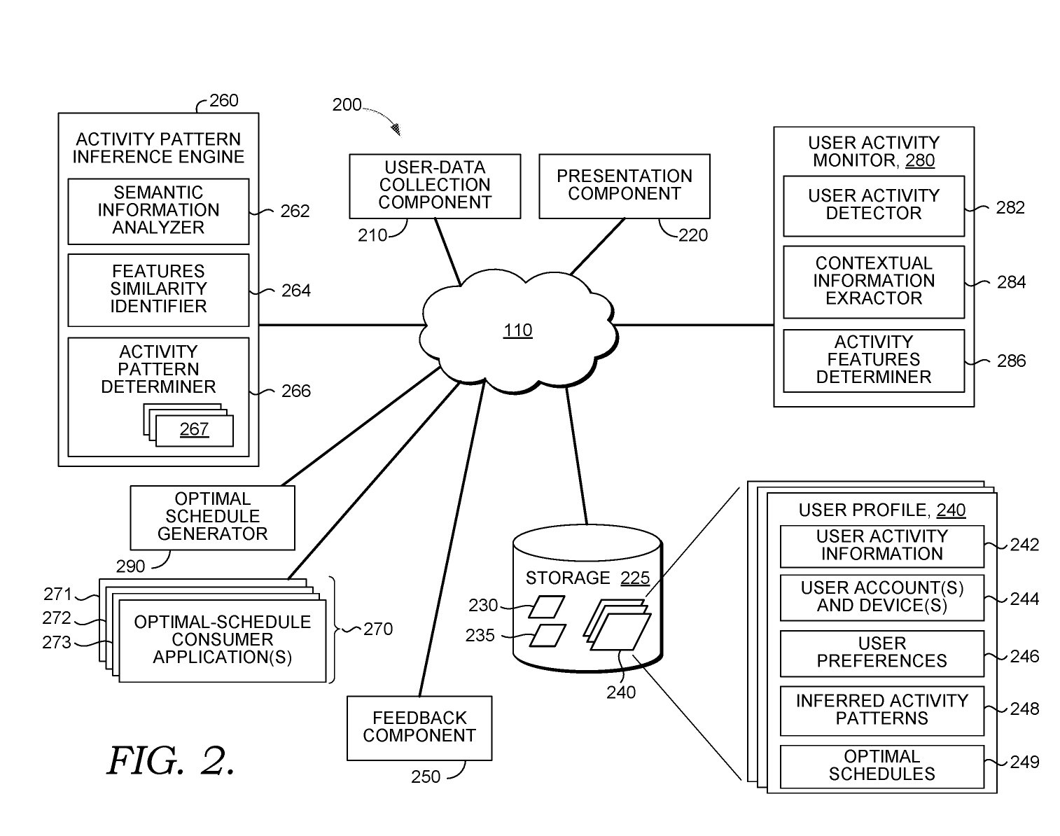

Accordingly, as will be described further herein, in one embodiment, user data is received from one or more data sources. The user data may be received by collecting user data with one or more sensors or other components on computing device(s) associated with the user, as described herein. In some embodiments, user data may include raw or unprocessed sensor data, interpretive data (as described herein), or data inferred from user interaction with a computing device, an application, or content. The received user data is used to determine or infer user activity information. The user data may also be used to determine contextual information related to the user activity information and likely future activities of the user. Examples of user data, which are further described in connection with component 210 of FIG. 2, may include information about the user computing device(s) (sometimes referred to herein as “user devices”), user activity associated with or determined by the user devices (e.g., user location data, user application (or app) related activity, such as application usage, which may include usage duration, launches, files accessed via the application or in conjunction with the application usage, or content associated with the application, online activity, searches, calls, usage duration, other user-interaction data, calendars, tasks, or other user activity as described herein), network-related data (such as network ID, connection data, or other network-related information), application data (e.g., meetings or calendar items, social network data, contacts data, email or communications data), or nearly any other source of user data that may be sensed or determined by a computing device. The received user data may be monitored and information about the user activity may be stored in a user profile, such as user profile 240 of FIG. 2. In some embodiments, user activity monitoring may be facilitated using an application or service that runs on the monitored user device. Alternatively, or in addition, the user activity monitoring may be facilitated using an application or service that runs in the cloud, which may scan the user device, or detect online activity associated with the user device, such as HTTP requests or other communication information, or otherwise receive information about user activity from the user device.

As further described herein, the user data may be used to determine or infer user activity, which may be monitored and used to determine user activity patterns. In some embodiments, the user activity monitor 280, which is generally responsible for receiving and monitoring user activity associated with one or more of the user's computing devices and/or applications, uses the user data provided from user-data collection component 210 to determine or infer user activity. For example, in one embodiment, user activity may be determined based on user data indicating a type of application content the user is interacting with, and/or how the user is interacting with the content (e.g., time spent on certain content, user-initiated changes to the content, amount, frequency, and duration of interruptions from the content that are initiated by the user, which could indicate a likelihood of whether the user is (or is not) involved in focus-intensive activity, or other user interactions). For instance, one aspect of monitored user activity may include a speed of scrolling through a document or other application content, highlighting text or otherwise designating portions of content in a document, zooming in or out of a document or other piece of content, an amount of eye movement a user makes, which may be determined from a webcam-type or camera-type sensor integrated into a computing device, and/or a frequency, duration, and use of a mouse or pointer to navigate or modify a document or other application content, for example. Additionally, some aspects of monitored user activity might include the frequency with which a user changes documents or other content (e.g., which may indicate a degree of focus), a duration of user-initiated interruptions (e.g., longer interruptions indicating a lower degree of focus), and/or periods of continuous interaction with content (e.g., representing continued engagement and focus).

Other aspects of user activity that may be determined from other user data include audio information or noise generated from the user or the user's environment (which may be detected using a microphone or audio sensor associated with a user computing device and which may be used to infer user activity and the type of user activity engaged in by the user). For example, silence or calm music may indicate focus-intensive work, while talking among multiple speakers may indicate collaborative work. Further, detected user activity may include user movement, which may be detected by a camera or other tracking device (which may also detect the user's physical presence), scrolling, activating, and/or deactivating computer applications, frequency and duration of activity or inactivity of the user, physiological information about the user, which may be detected using a wearable device (e.g., a fitness tracker), and which may be used to infer user activity or the type of user activity engaged in by the user, such as working out, stress-inducing work, relaxation, sleep, or the like.

User data may be analyzed to detect various features associated with user actions. Detected user actions, or “activity events,” may include actions such as websites visited, applications launched, working on a document, or other actions similar to those described herein, and may be logged with associated contextual data, for example, by logging the observed or detected user action with a corresponding time stamp, location stamp, and/or associating the activity event with other available contextual information (e.g., associated persons, computing devices, locations, related content (e.g., emails, calendar events, documents with similar subject matter, titles, files related to the same project, or other related content) or other contextual information). In some embodiments, such logging may be performed on each user device, so that user activity patterns may be determined or inferred across multiple devices. Further, in some embodiments, cloud-based user-activity information sources may be used, such as online user calendars or user activities determined from social media posts, emails, and the like. These sources also may be used for providing other context to the user activity detected on the user device(s). Examples of contextual information are further described in connection with contextual information extractor 284 depicted in FIG. 2. In some embodiments, user activity logs from multiple user devices and available user activity information from cloud-based sources may be combined, thereby representing a composite user activity history. The user activity logs, including corresponding contextual information, may be stored in a user profile associated with the user, such as user profile 240 shown in FIG. 2, as further described herein.

From the activity logs or user activity data, historical user activity information may be determined and provided to an inference engine. Based on an analysis of historical user activity, and in some cases current sensor data regarding user activity, a set of one or more user activity patterns may be determined. In particular, the inference engine may conduct an analysis of the historical user activity information to identify user activity patterns, which may be determined by detecting repetitions of similar user actions or routines, in some embodiments, or in other embodiments may be determined based on the time and duration of certain user activities, such as those that are similar (e.g., creating and/or modifying different types of content using an application).

In some embodiments, a corresponding confidence weight or confidence score may be determined for the user activity patterns. The confidence score may be based on the strength of the pattern, which may be determined by the number of observations used to determine a pattern, how frequently the user activity is consistent with the pattern, the age or freshness of the activity observations, the number of features in common with the activity observations that make up the pattern, or other similar measurements. In some instances, the confidence score may be considered when generating an optimal schedule. For instance, stronger patterns may be more influential (i.e., a generated optimal schedule may be more likely to include an allocation of time for a user activity or activity type indicated by a strong activity pattern; for instance, an optimal schedule may include time for eating lunch at noon where the user's pattern indicates the user nearly always eats lunch at that time). Further, in some embodiments, a minimum confidence score may be needed before using the activity pattern to generate an optimal schedule. For example, in one embodiment, a threshold of 0.6 (or just over fifty percent) is utilized such that only activity patterns having a 0.6 (or greater) likelihood of predicting user activity may be considered or used when generating an optimal schedule. In any case, when confidence scores and thresholds are used, determined patterns of user activity with confidence scores less than the threshold may still be monitored since additional observations of user activities may increase the confidence for a particular pattern.

In some embodiments, crowd-sourced user activity history may also be utilized in conjunction with a user's own activity history. For example, for a given user, a set of other users similar to the given user may be identified, based on having features or characteristics in common with the given user. This might include other users located in proximity to the given user, the given user's social media friends, users having the same employer, work colleagues (which may be determined from an analysis of contextual information associated with the given user), users of a similar age, profession, and/or interests (e.g., based on social media profiles), other users with similar user activity patterns, and the like. Information about user activity history from the other users may be relied upon for inferring patterns of user activity for a given user. This may be particularly useful in situations where little user activity history exists for the given user, such as where the user is a new user without an established record of user activity or established user activity patterns. In some embodiments, user activity information from similar users may be imputed to the new user until enough user history is available for the new user to determine statistically reliable user pattern predictions, which may be determined based on the number of observations included in the user activity history information or the statistical confidence of the determined user activity patterns, as further described herein. In some cases, where the user activity history comes from other users, the resulting inferred activity patterns for the given user may be assigned a lower confidence.

In some embodiments, a semantic analysis is performed on the determined or inferred user activity events and related information in order to characterize aspects of the user activity. For example, features associated with the user activity (or with an activity event) may be categorized (such as by type, similar time frame, similar location, similar computing device(s) or application(s), and the like, for example), and related features may be identified for use in determining a similarity or relational proximity to other activity events, such as by having one or more of the characteristics, including categories and related features, in common. In this way, the nature or type of user activity may be determined (e.g., focus-intensive work, reading, recreational activity, or other types of user activity). In some embodiments, a semantic knowledge representation, such as a relational knowledge graph, may be employed. In some embodiments, the semantic analysis may use rules, logic such as associations or conditions, and/or classifiers.

The semantic analysis may also be used to characterize information related to the user activity, such as determining that a location associated with the user activity corresponds to a hub or venue of interest to the user based on frequency of visits, such as the user's home, work, gym, a location frequently traveled to, such as for work or pleasure, etc. For example, a user's home hub may be determined to be the location where the user spends most of his or her time between 8 PM and 6 AM. Similarly, the semantic analysis may determine the time of day that corresponds to working hours, lunch time, commuting time, sleeping time, or the like. In this way, the semantic analysis may provide other relevant features of the activity events that may be used to determine activity patterns. For example, in addition to determining a particular website that the user visited at a certain time, such as visiting CNN.com during a user's lunch break, the category of the website may be determined, which in this case, for example, might be a news-related website. Similarly, the semantic analysis may categorize the activity as being associated with work or home, based on the characteristics of the activity (e.g., a batch of online searches about chi-squared distribution that occurs during working hours at a location corresponding to the user's office may be determined to be work-related activity, and in contrast, streaming a movie on a Friday night at a location corresponding to the user's home may be determined to be home-related activity or recreational activity). These aspects characterizing the user activity event may then be considered when evaluating the user activity history to identify patterns.

Based on the activity patterns of the user, future user activity and/or types of future user activity may be inferred, and one or more optimal schedules of user activity may be generated. Future user activity may be classified as an activity type. In some embodiments, user preferences and/or user feedback also may be utilized when generating optimal schedules, as further described below. In some embodiments, an optimal schedule may include an allocation of time specifying a future user activity or type of user activity. For example, an optimal schedule may include allocations of time for types of user activities such as focus-intensive work, non-focus-intensive work, multitasking, recreational activity, traveling, sleeping, meetings/collaborative work, communicating or responding to communications, planning, organizing and preparing, reading, writing, coding, programming, calculating, analyzing, creating or editing content, or other types of user activity. The optimal schedule(s) may be provided to one or more computerized personal assistant tools, such as the optimal-schedule consumer applications 270 described herein, which utilize the schedule information to provide an enhanced user experience. In particular, three examples of optimal-schedule consumer applications, which are further described below, include a calendar management program, which utilizes an optimal schedule for managing a user's calendar events to be more in accordance with the optimal schedule, a personal performance optimizer application, which utilizes an optimal schedule to provide the user with a recommended daily schedule of user activity based on future activity to be performed by the user, and a notifications service, which utilizes an optimal schedule to manage electronic notifications to the user or to other people.

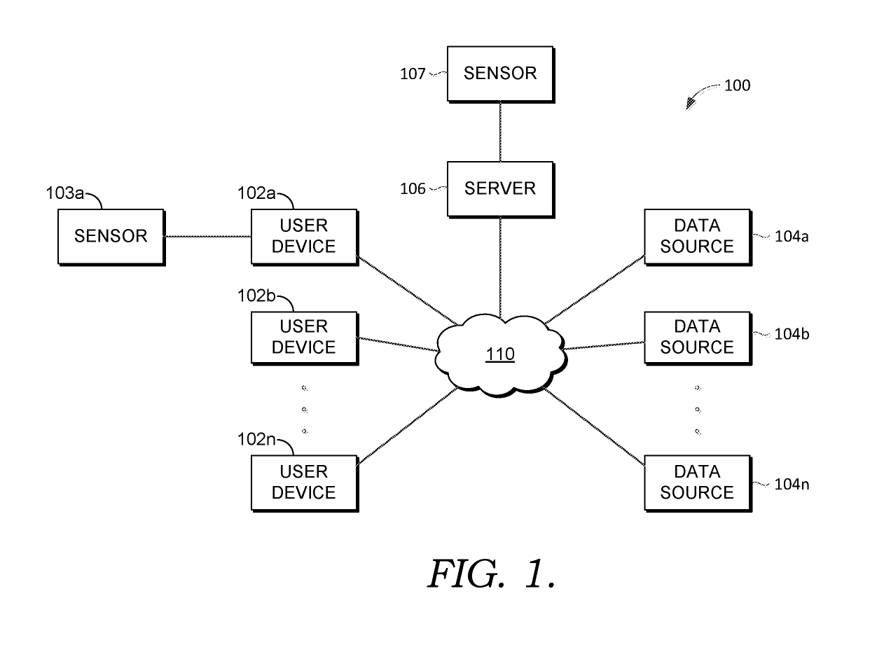

Turning now to FIG. 1, a block diagram is provided showing an example operating environment 100 in which some embodiments of the present disclosure may be employed. It should be understood that this and other arrangements described herein are set forth only as examples. Other arrangements and elements (e.g., machines, interfaces, functions, orders, and groupings of functions, or other arrangements or elements) may be used in addition to, or instead of, those shown, and some elements may be omitted altogether for the sake of clarity. Further, many of the elements described herein are functional entities that may be implemented as discrete or distributed components, or in conjunction with other components, and in any suitable combination and location. Various functions described herein as being performed by one or more entities may be carried out by hardware, firmware, and/or software. For instance, some functions may be carried out by a processor executing instructions stored in memory, whether locally or in the cloud.

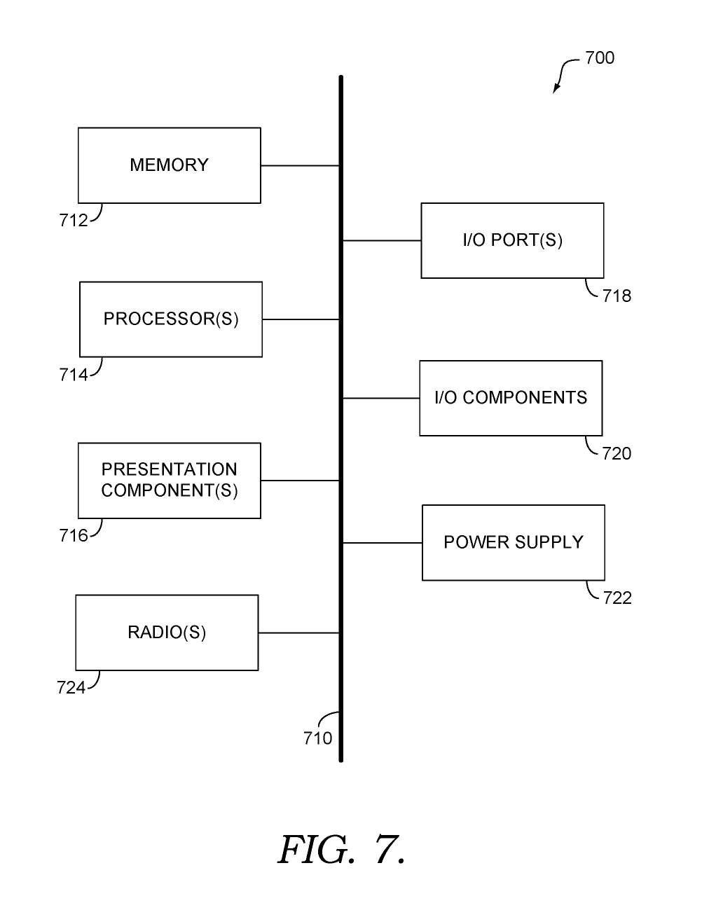

Among other components not shown, example operating environment 100 includes a number of user devices, such as user devices 102a and 102b through 102n; a number of data sources, such as data sources 104a and 104b through 104n; server 106; sensors 103a and 107; and network 110. It should be understood that operating environment 100 shown in FIG. 1 is an example of one suitable operating environment. Each of the components shown in FIG. 1 may be implemented via any type of computing device, such as computing device 700 described in connection with FIG. 7, for example. These components may communicate with each other via network 110, which may include, without limitation, one or more local area networks (LANs) and/or wide area networks (WANs). In exemplary implementations, network 110 comprises the Internet and/or a cellular network, amongst any other of a variety of possible public and/or private networks.

It should be understood that any number of user devices, servers, and data sources may be employed within operating environment 100 and these should be considered within the scope of the present technology. Each may comprise a single device or multiple devices cooperating in a distributed environment. For example, server 106 may be provided via multiple devices arranged in a distributed environment that collectively provide the functionality described herein. Additionally, other components not shown may also be included within the distributed environment.

User devices 102a and 102b through 102n may be client devices on the client-side of operating environment 100, while server 106 may be on the server-side of operating environment 100. Server 106 can comprise server-side software designed to work in conjunction with client-side software on user devices 102a and 102b through 102n so as to implement any combination of the features and functionalities discussed in the present disclosure. This division of operating environment 100 is provided to illustrate one example of a suitable environment, and there is no requirement for each implementation that any combination of server 106 and user devices 102a and 102b through 102n remain as separate entities.

User devices 102a and 102b through 102n may comprise any type of computing device capable of use by a user. For example, in one embodiment, user devices 102a through 102n may be the type of computing device described in relation to FIG. 7 herein. By way of example and not limitation, a user device may be embodied as a personal computer (PC), a laptop computer, a mobile or mobile device, a smartphone, a tablet computer, a smart watch, a wearable computer, a personal digital assistant (PDA), an MP3 player, a global positioning system (GPS) or device, a video player, a handheld communications device, a gaming device or system, an entertainment system, a vehicle computer system, an embedded system controller, a camera, a remote control, an appliance, a consumer electronic device, a workstation, or any combination of these delineated devices, or any other suitable device.

Data sources 104a and 104b through 104n may comprise data sources and/or data systems, which are configured to make data available to any of the various constituents of operating environment 100 or system 200 described in connection with FIG. 2. For example, in one embodiment, one or more data sources 104a through 104n provide, or make available for accessing, user data to user-data collection component 210 of FIG. 2. Data sources 104a and 104b through 104n may be discrete from user devices 102a and 102b through 102n and server 106, or may be incorporated and/or integrated into at least one of those components. In one embodiment, one or more of data sources 104a through 104n comprise one or more sensors, which may be integrated into or associated with one or more of the user device(s) 102a, 102b, or 102n, or server 106. Examples of sensed user data made available by data sources 104a through 104n are described further in connection to user-data collection component 210 of FIG. 2.

Operating environment 100 can be utilized to implement one or more of the components of system 200, described in FIG. 2, including components for collecting user data, monitoring activity events, determining activity patterns, determining an optimal schedule, receiving user feedback or preferences, and consuming optimal schedule information to provide an enhanced user experience, which may include managing a user's calendar or personal schedule, generating personalized content, and/or managing notifications on behalf of the user.

Referring now to FIG. 2, with FIG. 1, a block diagram is provided showing aspects of an exemplary computing system architecture suitable for implementing an embodiment of the present technology, designated generally as system 200. System 200 represents only one example of a suitable computing system architecture. Other arrangements and elements can be used in addition to, or instead of, those shown, and some elements may be omitted altogether for the sake of clarity. Further, as with operating environment 100, many of the elements described herein are functional entities that may be implemented as discrete or distributed components or in conjunction with other components, and in any suitable combination and location.

Example system 200 includes network 110, which is described in connection to FIG. 1, and which communicatively couples components of system 200 including user-data collection component 210, presentation component 220, user activity monitor 280, activity pattern inference engine 260, optimal schedule generator 290, optimal-schedule consumer applications 270 including exemplary applications 271-273, feedback component 250, and storage 225. User activity monitor 280 (including its subcomponents 282, 284, and 286), activity pattern inference engine 260 (including its subcomponents 262, 264, 266, and 267), user-data collection component 210, presentation component 220, optimal schedule generator 290, optimal-schedule consumer applications 270 including exemplary applications 271-273, and feedback component 250 may be embodied as a set of compiled computer instructions or functions, program modules, computer software services, or an arrangement of processes carried out on one or more computer systems, such as computing device 700 described in connection with FIG. 7, for example.

In one embodiment, functions performed by components of system 200, such as the optimal-schedule consumer applications 270 including applications 271-273, are associated with one or more personal assistant applications, services, and/or routines. In particular, such applications, services, or routines may operate on one or more user devices (such as user device 102a), servers (such as server 106), may be distributed across one or more user devices and servers, and/or may be implemented in the cloud. Moreover, in some embodiments, these components of system 200 may be distributed across a network, including across one or more servers (such as server 106) and/or client devices (such as user device 102a), in the cloud, or may reside on a user device, such as user device 102a. Moreover, these components, functions performed by these components, or services carried out by these components may be implemented at appropriate abstraction layer(s), such as the operating system layer, application layer, or hardware layer of the computing system(s). Alternatively, or in addition, the functionality of these components and/or the embodiments described herein can be performed, at least in part, by one or more hardware logic components. For example, and without limitation, illustrative types of hardware logic components that can be used include Field-programmable Gate Arrays (FPGAs), Application-specific Integrated Circuits (ASICs), Application-specific Standard Products (ASSPs), System-on-a-chip systems (SOCs), and Complex Programmable Logic Devices (CPLDs). Additionally, although functionality is described herein with regards to specific components shown in example system 200, it is contemplated that in some embodiments functionality of these components can be shared or distributed across other components.

Continuing with FIG. 2, user-data collection component 210 is generally responsible for receiving and/or obtaining (and in some cases also identifying) user data from one or more data sources, such as data sources 104a and 104b through 104n of FIG. 1. In some embodiments, user-data collection component 210 may be employed to facilitate the accumulation of user data of a particular user (or in some cases, a plurality of users, including crowdsourced data) that is provided to user activity monitor 280, activity pattern inference engine 260, or optimal-schedule consumer application 270, including exemplary applications 271-273. The data may be received by the user-data collection component 210 (or accessed), and optionally accumulated, reformatted, and/or combined by user-data collection component 210 and stored in one or more data stores, such as in the user activity profile 240 associated with storage 225, where it may be made available or accessible to other components of system 200. For example, the user data may be stored in or associated with user profile 240, as described herein. In some embodiments, any personally identifying data (i.e., user data that specifically identifies particular users) is either not uploaded or otherwise provided separately from the one or more data sources with user data, is not permanently stored, and/or is not made available to user activity monitor 280 and/or activity pattern inference engine 260.

User data, including user data received or obtained by the user-data collection component 210, may be from a variety of sources where the data may be available in a variety of formats. For example, in some embodiments, user data received via user-data collection component 210 may be determined via one or more sensors (such as sensors 103a and 107 of FIG. 1), which may be on or associated with one or more user devices (such as user device 102a), servers (such as server 106), and/or other computing devices. As used herein, a sensor may include a function, routine, component, or combination thereof for sensing, detecting, or otherwise obtaining information such as user data from a data source 104a, and may be embodied as hardware, software, or both.

Further, by way of example, and not limitation, user data may include data that is sensed or determined by one or more sensors (referred to herein as sensor data), such as location information of mobile device(s), properties or characteristics of user device(s) (e.g., active and in use or inactive and not in use), user-activity information (for example: application usage, online activity, searches, voice data such as automatic speech recognition, activity logs, communications data including calls, texts, instant messages, and emails, website posts, other user data associated with communication events, or other types of user activity information) including, in some embodiments, user activity that occurs over more than one user device, user history, session logs, application data, contacts data, calendar and schedule data, notification data, and social-network data, user-account(s) data (which may include data from user preferences provided from user preferences 246 or feedback component 250, or settings associated with a personal assistant application or service), home-sensor data, appliance data, global positioning system (GPS) data, vehicle signal data, traffic data, weather data (including forecasts), physiological information about the user (which may be determined from a wearable device), other wearable device data, other user device data (which may include user motion or ambulation data, device settings, profiles, network-related information (e.g., network name or ID, domain information, workgroup information, connection data, Wi-Fi network data, or configuration data, data regarding model number, firmware, or equipment, device pairings, such as where a user has a mobile phone paired with a Bluetooth speaker or headset, for example, or other network-related information)), gyroscope data, accelerometer data, payment or credit card usage data (which may include information from a user's PayPal® account or another finance-related account), purchase history data (such as information from a user's Xbox Live, Amazon.com, or eBay account), other sensor data that may be sensed or otherwise detected by a sensor (or other detector) component(s) including data derived from a sensor component associated with the user (including location, motion, orientation, position, user-access, user-activity, network-access, user-device-charging, or other data that is capable of being provided by one or more sensor components), data derived based on other data (for example, location data that can be derived from Wi-Fi, cellular network, or IP address data), and nearly any other source of data that may be sensed or determined as described herein.

In some respects, user data may be provided in user-data streams or signals. A “user signal” can be a feed or stream of user data from a corresponding data source. For example, a user signal could be from a smartphone, a home-sensor device, a GPS device (e.g., for location coordinates), a vehicle-sensor device, a wearable device, a user device, a gyroscope sensor, an accelerometer sensor, a calendar service, an email account, a credit card account, or other data sources. In some embodiments, user-data collection component 210 receives or accesses data continuously, periodically, or as needed.

In some embodiments, a sensor may include software and/or hardware that enables the sensor to collect user activity data, including physical activity of the user and/or data related to interactivity between the user and the user's computing devices and applications. For example, one or more sensors may be used to detect and provide physical and/or biometric data of the user during user activity, such as eye movement, body movement, pulse, brain activity, voice or speech activity, and the like using sensors configured to collect data on the same, any of which may be used to identify or characterize the nature of user activity (e.g., focused, non-focused/distracted, collaborative, recreational, or other activity types described herein). In this respect, a sensor may be configured to track the activity of the user using implements such as an eye-movement or body-movement tracking camera, a pointer (e.g., mouse) tracker, a keyboard tracker, a speech or voice tracker (e.g., using one or more microphones), and the like, indicating what type of activity the user is engaged in, and the level of engagement (e.g., based on frequency of interaction, intensity (e.g., volume or speed of movement), or duration, for example).

In other embodiments, a sensor may include software and/or hardware that enables the sensor to collect data on interactions between the user and the user's computing devices and applications that can be used by other components of the system 200 to determine the level of user engagement or distraction. For example, a sensor may be able to detect data indicating a frequency and duration of sustained interaction between a user and an application or content (e.g., allowing a level of user engagement to be determined or inferred by other components that receive the data, such as user activity monitor 280 or activity pattern inference engine 260, for example), may be able to detect data indicating a frequency and duration of changes in applications or content by the user (e.g., allowing a level of user distraction to be determined or inferred by other components that receive the data, such as user activity monitor 280 or activity pattern inference engine 260, for example), and/or may be able to provide information from which it may be determined how the user interacts with disruptions (e.g., how often and for how long a user engages with a disrupting event). With respect to disruptions, a sensor may be able to detect characteristics of a disruption (e.g., the type of disruption, such as user-initiated, non-user initiated, or other characteristics), and data indicating how a user interacts with the disruption (e.g., opens an application, notification, or message, or engages with a different application, or ignores the disruption and continues working, or other interactions).

In some embodiments, a sensor may be configured to detect the way in which a user interacts with content or an application. For example, a speed with which a user reads, writes, or modifies data or content within an application may be detected by a sensor, so that other components of the system 200, such as the user activity monitor 280, can use the data collected by the sensor to determine a level of user focus or engagement (e.g., a slower speed indicating more engagement and focus, versus quickly skimming or browsing the content indicating less engagement and focus). Further, a sensor may be configured to detect a type of modification of content, such as editing, highlighting, zooming, copying, and/or revising of the content. In some embodiments, a sensor may be configured to detect a duration of disruption where a user is not engaged with content. For example, if a user is focused on content and is interrupted and diverts away from the content, a duration of the interruption may be measured (e.g., 3, 5, 10, 20, or 30 seconds, for example).

User activity monitor 280 is generally responsible for monitoring user data for information that may be used for determining user activity information, which may include identifying and/or tracking features (sometimes referred to herein as “variables”) or other information regarding specific user actions and related contextual information. Embodiments of user activity monitor 280 may determine, from the monitored user data, user activity associated with a particular user. In some embodiments, user activity monitor 280 uses the user data received from user-data collection component 210 to determine or infer user activity. For example, in one embodiment, user activity may be determined based on user data indicating what content the user is interacting with (e.g., a word document, a messaging application, a website, or other content), and/or how the user is interacting with the content (e.g., time spent on certain content, user-initiated changes to the content, amount, frequency, and duration of interruptions from the content that are initiated by the user, or other user interactions). For example, activity monitoring by the user activity monitor 280 may include determining a speed of scrolling through a document or other content, highlighting text or other portions of content in a document, zooming in or out of a document or other content, an amount of eye movement a user makes, which may be determined from a webcam-type or camera-type sensor integrated into a computing device, and/or a frequency, duration, and use of a mouse or pointer to navigate or modify a document or other content, for example. Further, activity monitoring may be used to indicate the nature or type of activity the user is engaged in, such as focus-intensive or non-focus-intensive activity.

Specifically, in some embodiments, user activity monitor 280 may monitor user activity and associated activity features to determine a level of engagement or focus associated with the user. For example, some aspects of monitored user activity might include the frequency with which a user changes documents or other content (e.g., indicating a degree of engagement and focus), a duration of disruptions (e.g., longer or more frequent interruptions indicating less focus), and/or periods of continuous interaction with a particular piece of content (e.g., representing continued engagement and focus).

Other aspects of user activity that may be determined from other user data include audio information or noise generated from the user or the user's environment (e.g., detected using a microphone or audio sensor associated with a user computing device, which may be used to infer user activity and/or the type of user activity engaged in by the user; for example, silence or calm music may indicate focus-intensive work or talking among multiple detected speakers may indicate collaborative work); user movement, which may be detected by a camera or other tracking device (which may also detect the user's physical presence); scrolling, activating, and/or deactivating computer applications; frequency and duration of activity or inactivity of the user; physiological information about the user (which may be detected using a wearable device (e.g., a fitness tracker), and which may be used to infer user activity or the type of user activity engaged in by the user, such as working out, stress-inducing work, relaxation, sleep, or the like).

The user activity information determined by user activity monitor 280 may include user activity information from multiple user devices associated with the user and/or from cloud-based services associated with the user (e.g., email, calendars, social-media, or similar information sources), and may include contextual information associated with the identified user activity. User activity monitor 280 may determine current or near-real-time user activity information and may also determine historical user activity information, in some embodiments, which may be determined based on gathering observations of user activity over time, accessing user logs of past activity (such as browsing history, for example), and accessing stored user activity data, such as from stored activity user information 242 in user profile 240. Further, in some embodiments, user activity monitor 280 may determine user activity (which may include historical activity) from other similar users (i.e., crowdsourcing), as described previously.

In some embodiments, information determined by user activity monitor 280 may be provided to activity pattern inference engine 260 including information regarding current context and historical activity (historical observations). Some embodiments may also provide user activity information, such as current user activity, to optimal-schedule consumer applications 270, including exemplary applications 271-273. As described previously, user activity features may be determined by monitoring user data received from user-data collection component 210. In some embodiments, the user data and/or information about the user activity determined from the user data is stored in a user profile, such as in user activity information 242 of user profile 240.

In an embodiment, user activity monitor 280 comprises one or more applications or services that analyze information detected via one or more user devices used by the user and/or cloud-based services associated with the user, to determine activity information and related contextual information. Information about user devices associated with a user may be determined from the user data made available via user-data collection component 210, and may be provided to user activity monitor 280, activity pattern inference engine 260, or other components of system 200. More specifically, in some implementations of user activity monitor 280, a user device may be identified by detecting and analyzing characteristics of the user device, such as device hardware, software, such as the user device's operating system (OS), network-related characteristics, user accounts accessed via the device, and similar characteristics. For example, information about a user device may be determined using functionality of many operating systems to provide information about the hardware, OS version, network connection information, installed application(s), or the like.

Some embodiments of user activity monitor 280, or its sub-components, may determine a device name or device identification (device ID) for each device associated with a user. This information about the identified user devices associated with a user may be stored in a user profile associated with the user, such as in user account(s) and device(s) 244 of user profile 240. In an embodiment, the user devices may be polled, interrogated, or otherwise analyzed to determine information about the devices. This information may be used for determining a label or identification for the device (e.g., a device ID) so that user interaction with the device may be recognized from user data by user activity monitor 280. In some embodiments, users may declare or register a device, such as by logging into an account via the device, installing an application on the device, connecting to an online service that interrogates the device, or otherwise providing information about the device to an application or service. In some embodiments, devices that sign into an account associated with the user, such as a Microsoft® account or Net Passport, email account, social network account, and the like, can be identified and determined to be associated with the user.

As shown in example system 200, user activity monitor 280 comprises a user activity detector 282, a contextual information extractor 284, and an activity features determiner 286. In some embodiments, user activity monitor 280, one or more of its sub-components, or other components of system 200, such as optimal-schedule consumer applications 270 including applications 271-273 or activity pattern inference engine 260, may determine interpretive data from received user data. Interpretive data corresponds to data utilized by these components of system 200 or sub-components of user activity monitor 280 to interpret user data. Interpretive data can be used to provide other context to user data, which can support determinations or inferences made by the components or sub-components. For example, interpretive data may include data indicating a user's speed (which may be used to infer that the user is traveling) based on raw sensor user data indicating user location information. Moreover, it is contemplated that embodiments of user activity monitor 280, its sub-components, and other components of system 200 may use user data and/or user data in combination with interpretive data for carrying out the objectives of the sub-components described herein. Additionally, although several examples of how user activity monitor 280 and its sub-components may identify user activity information are described herein, many variations of user activity identification and user activity monitoring are possible in various embodiments of the disclosure.

User activity detector 282, in general, is responsible for determining (or identifying) that a user action or activity event has occurred. Embodiments of user activity detector 282 may be used for determining current user activity or one or more historical user actions or activities. Some embodiments of activity detector 282 may monitor user data for activity-related features or variables corresponding to user activity such as indications of applications launched or accessed, files accessed, modified, or copied, websites navigated to, online content downloaded and rendered or played, user devices used, locations visited, the presence of other people detected in proximity to the user, user activity indicated by physiological information, communications (e.g., calls, chats, emails, or the like) performed by the user, or other user activities that may be detected or inferred from the user data.

Additionally, some embodiments of user activity detector 282 extract from the user data information about user activity, which may include current user activity, historical user activity, and/or related information, such as contextual information. Alternatively, or in addition, in some embodiments, contextual information extractor 284 determines and extracts or infers contextual information, and similarly, in some embodiments, activity features determiner 286 extracts or infers information about user activity, such as user activity-related features, based on an identification of the activity determined by user activity detector 282. Examples of extracted or inferred user activity information may include application usage, online activity, searches, calls, usage duration, application data (e.g., emails, messages, posts, user status, notifications, or other application data), information characterizing the nature or type of activity (e.g., work-related activity, personal-related activity, focus-intensive or non-focus-intensive work, multitasking, recreational activity, traveling, sleeping, meetings/collaborative work, communicating or responding to communications, planning, organizing and preparing, reading, writing, coding, programming, calculating, analyzing, creating or editing content, or other types of user activity) or nearly any other data related to user interactions with the user device or user activity via a user device. Among other components of system 200, the extracted or inferred user activity information determined by user activity detector 282 may be provided to other sub-components of user activity monitor 280, activity pattern inference engine 260, or optimal-schedule consumer applications 270. Further, the extracted user activity may be stored in a user profile associated with the user, such as in user activity information 242 of user profile 240. In some embodiments, user activity detector 282 or user activity monitor 280 (or its other sub-components) performs conflation on the detected user activity information. For example, overlapping information may be merged and duplicated or redundant information eliminated.

In some embodiments, the user activity-related features may be interpreted to determine a user activity has occurred. For example, in some embodiments, user activity detector 282 employs user activity event logic, which may include rules, conditions, associations, classification models, or other criteria, to identify user activity. For example, in one embodiment, user activity event logic may include comparing user activity criteria with the user data in order to determine that an activity event has occurred. The activity event logic can take many different forms depending on the mechanism used to identify an activity event. For example, the user activity event logic may include training data used to train a neural network that is used to evaluate user data to determine when an activity event has occurred. The activity event logic may comprise fuzzy logic, neural network, finite state machine, support vector machine, logistic regression, clustering, or machine-learning techniques, similar statistical classification processes, or combinations of these to identify activity events from user data. For example, activity event logic may specify types of user device interaction(s) information that are associated with an activity event, such as navigating to a website, composing an email, or launching an application. In some embodiments, a series or sequence of user device interactions may be mapped to an activity event, such that the activity event may be detected upon determining that the user data indicates the series or sequence of user interactions has been carried out by the user.

In some embodiments, activity event logic may specify types of user-device-related activity that is considered user activity, such as activity that happens while a user is logged into the user device, while user interfaces are receiving input (e.g., while a computer mouse, touchpad, screen, voice-recognition interface, or the like are active), or certain types of activity like launching applications, modifying files with applications, opening a browser and navigating to a website, or other types of activity. In this way, the activity event logic may be used to distinguish genuine user activity from automated activity of processes running on the user devices, such as automatic updates or malware scanning. Once a user activity is determined, these features or additional related features may be detected and associated with the detected activity for use in determining activity patterns.

In some embodiments, user activity detector 282 runs on or in association with each user device of a user. User activity detector 282 may include functionality that polls or analyzes aspects of the operating system to determine user activity-related features (such as installed or running applications or file accesses and modifications, for example), network communications, and/or other user actions detectable via the user device including sequences of actions.

Contextual information extractor 284, in general, is responsible for determining contextual information related to the user activity (detected by user activity detector 282 or user activity monitor 280), such as context features or variables associated with user activity, related information (e.g., location or time of the activity), and user-related activity, and further is responsible for associating the determined contextual information with the detected user activity. In some embodiments, contextual information extractor 284 may associate the determined contextual information with the related user activity and may also log the contextual information with the associated user activity. Alternatively, the association or logging may be carried out by another service. For example, some embodiments of contextual information extractor 284 provide the determined contextual information to activity features determiner 286, which determines activity features of the user activity and/or related contextual information. Some embodiments of contextual information extractor 284 may also be used to determine a current context for a user based on user data received from user-data collection component 210 and/or user activity determined by user activity monitor 280.

Some embodiments of contextual information extractor 284 determine contextual information related to a user action or activity event such as entities identified in a user activity or related to the activity (e.g., recipients of a group email sent by the user), user activity associated with the location or venue of the user's device, or other contextual information. By way of example and not limitation, this may include context features such as location data, which may be represented as a location stamp associated with the activity; contextual information about the location, such as venue information (e.g., this is the user's office location, home location, school, restaurant, movie theater, or another venue), yellow pages identifier (YPID) information, time, day, and/or date, which may be represented as a time stamp associated with the activity; user device characteristics or user device identification information regarding the device on which the user carried out the activity; duration of the user activity, other user activity/activities preceding and/or following the user activity (such as sequences of websites visited, a sequence of online searches conducted, sequences of application and website usage, such as browsing to a bank and then accessing an Excel® spreadsheet file to record financial information, or the like), other information about the activity such as entities associated with the activity (e.g., venues, people, objects, or other entities), information detected by sensor(s) on user devices associated with the user that is concurrent or substantially concurrent with the user activity (e.g., motion information or physiological information detected on a fitness tracking user device, listening to music, which may be detected via a microphone sensor if the source of the music is not a user device), or any other information related to the user activity that is detectable that may be used for determining patterns of user activity.

In embodiments, using contextual information related to user devices, a user device may be identified by detecting and analyzing characteristics of the user device, such as device hardware, software such as operating system (OS), network-related characteristics, user accounts accessed via the device, and similar characteristics. For example, as described previously, information about a user device may be determined using functionality of many operating systems to provide information about the hardware, OS version, network connection information, installed application, or the like. In some embodiments, a device name or identification (device ID) may be determined for each device associated with a user. This information about the identified user devices associated with a user may be stored in a user profile associated with the user, such as in user account(s) and device(s) 244 of user profile 240. In an embodiment, the user devices may be polled, interrogated, or otherwise analyzed to determine contextual information about the devices. This information may be used for determining a label or identification of the device (e.g., a device ID) so that user activity on one user device may be recognized and distinguished from user activity on another user device. Further, as described previously, in some embodiments, users may declare or register a user device, such as by logging into an account via the device, installing an application on the device, connecting to an online service that interrogates the device, or otherwise providing information about the device to an application or service. In some embodiments, devices that sign into an account associated with the user, such as a Microsoft® account or MSA (previously known as Net Passport), email account, social network, or the like, are identified and determined to be associated with the user.

In some implementations, contextual information extractor 284 may receive user data from user-data collection component 210, parse the data, in some instances, and identify and extract context features or variables (which may also be carried out by activity features determiner 286). Context variables may be stored as a related set of contextual information associated with the user activity, and may be stored in a user profile, such as in user activity information component 242. In some cases, contextual information may be used by one or more activity pattern consumers, such as for personalizing content or a user experience, such as when, where, or how to present content. Contextual information may also be determined from the user data of one or more users, in some embodiments, which may be provided by user-data collection component 210 in lieu of or in addition to user activity information for the particular user.

Activity features determiner 286 is generally responsible for determining activity-related features (or variables) associated with the user activity that may be used for identifying patterns of user activity. Activity features may be determined from information about a user activity and/or from related contextual information. In some embodiments, activity features determiner 286 receives user activity or related information from user activity monitor 280 (or its sub-components), and analyzes the received information to determine a set of one or more features associated with the user activity.

Examples of activity-related features include, without limitation, location-related features, such as location of the user device(s) during the user activity, venue-related information associated with the location, or other location-related information; time-related features, such as time(s) of day(s), day of week or month, or the duration of the activity, or related duration information such as how long the user used an application associated with the activity; user device-related features, such as device type (e.g., desktop, tablet, mobile phone, fitness tracker, heart rate monitor, or other types of devices), hardware properties or profiles, OS or firmware properties, device IDs or model numbers, network-related information (e.g., mac address, network name, IP address, domain, work group, information about other devices detected on the local network, router information, proxy or VPN information, or other network connection information), position/motion/orientation-related information about the user device, power information such as battery level, time of connecting/disconnecting a charger, and user-access/touch information; usage-related features, such as file(s) accessed, application usage (which may also include application data, in-app usage, concurrently running applications), network usage information, user account(s) accessed or otherwise used (such as device account(s), OS level account(s), or online/cloud-services related account(s) activity, such as Microsoft® account or Net Passport activity, online storage account(s), email, calendar, or social networking accounts, or other user accounts); content-related features, such as online activity (e.g., searches, browsed websites, purchases, social networking activity, communications sent or received, including social media posts); other features that may be detected concurrent with the user activity or near the time of the user activity; or any other features that may be detected or sensed and used for determining a pattern of the user activity. Features may also include information about user(s) using the device; other information identifying a user, such as a login password, biometric data, which may be provided by a fitness tracker or biometric scanner (e.g., fingerprint scanner or facial feature recognizer); and/or characteristics of the user(s) who use the device, which may be useful for distinguishing users on devices that are shared by more than one user. In some embodiments, user activity event logic (described in connection with user activity detector 282) may be utilized to identify specific features from user activity information. In some embodiments, a set of activity features associated with an activity event may be stored in user activity information 242 of user profile 240 (on storage 225).

Continuing with system 200 of FIG. 2, activity pattern inference engine 260 is generally responsible for determining user activity patterns based on the user activity information determined from user activity monitor 280. In some embodiments, activity pattern inference engine 260 may run on a server, as a distributed application across multiple devices, or in the cloud. At a high level, activity pattern inference engine 260 may receive user activity-related information, which may be uploaded from user activity logs from client-side applications or services associated with user activity monitor 280. One or more inference algorithms may be applied to the user activity-related information to determine a set of user activity patterns, including current and/or historical patterns. For example, activity patterns may be determined based on similar instances of observation of user activity or associated contextual information, which may be referred to as “in-common features” of user activity-related information. For example, identified user activities that are performed with regularity, such as those performed at regular times, time intervals, locations, or in certain circumstances, may be considered to include the “in-common features.”

As shown in example system 200, activity pattern inference engine 260 comprises semantic information analyzer 262, features similarity identifier 264, and activity pattern determiner 266. Semantic information analyzer 262 is generally responsible for determining semantic information associated with the user activity-related features identified by user activity monitor 280. For example, while a user-activity feature may indicate a specific type of content being modified by the user using an application on the user's computing device, semantic analysis may determine the type or nature of activity performed by the user, such as focus-intensive work, non-focus-intensive work, multitasking, recreational activity, traveling, sleeping, meetings/collaborative work, communicating or responding to communications, planning, organizing and preparing, reading, writing, coding, programming, calculating, analyzing, creating or editing content, or other types of user activity. Semantic information analyzer 262 may determine additional user activity-related features semantically related to the user activity, which may be used for identifying user activity patterns.

In particular, as described previously, a semantic analysis may be performed on the user activity information, which may include the contextual information, to characterize aspects of the user action or activity event. For example, in some embodiments, activity features associated with an activity event may be classified or categorized (such as by activity type, time frame or location, work-related, home-related, themes, related entities, other user(s) (such as communication to or from another user) and/or relation of the other user to the user (e.g., family member, close friend, work acquaintance, boss, or the like), or other categories), or related features may be identified for use in determining a similarity or relational proximity to other user activity events, which may indicate a pattern. In some embodiments, semantic information analyzer 262 may utilize a semantic knowledge representation, such as a relational knowledge graph. Semantic information analyzer 262 may also utilize semantic analysis logic, including rules, conditions, or associations to determine semantic information related to the user activity.

For example, a user activity event comprising an email sent to someone who works with the user may be characterized as a work-related activity. Thus, where the user emails some person she works with every Sunday night, but not necessarily the same person, a pattern may be determined (using activity pattern determiner 266) that the user performs work-related activities on Sunday nights. Similarly, an activity event comprising the user opening a computer application (such as a word processor program or spreadsheet), writing to or modifying a document via the computing application, along with other user data (e.g., scroll speed, mouse movement, or the like) may be characterized as focus-intensive work. Thus, where the user performs this type of activity for several hours every workday morning, but the particular computer application or document may change, a pattern may be determined that the user performs focus-intensive activity on workday mornings. Accordingly, it may be appropriate to allocate time for this work-related activity for an appropriate time (e.g., on each Sunday night for the email pattern or for workday mornings for the focus-intensive activity pattern), in an optimal schedule that is generated for the user by optimal schedule generator 290, which may be based on the user activity pattern determined or inferred by the activity pattern inference engine 260, as discussed below with respect to optimal schedule generator 290.

Semantic information analyzer 262 may be used to characterize contextual information associated with the user activity event, such as determining that a location associated with the activity corresponds to a hub or venue of interest to the user (such as the user's home, work, gym, or the like) based on frequency of user visits. For example, the user's home hub may be determined (using semantic analysis logic) to be the location where the user spends most of her time between 8 PM and 6 AM. Similarly, the semantic analysis may determine time of day that corresponds to working hours, lunch time, commute time, or other times of day. Similarly, the semantic analysis may categorize the activity as being associated with work or home, based on other characteristics of the activity (e.g., a batch of online searches about chi-squared distribution that occurs during working hours at a location corresponding to the user's office may be determined to be work-related activity, and streaming a movie on Friday night at a location corresponding to the user's home may be determined to be home-related or recreational activity). In this way, the semantic analysis provided by semantic information analyzer 262 may provide other relevant features of the user activity events that may be used for determining user activity patterns.

Features similarity identifier 264 is generally responsible for determining the similarity of activity features of two or more user activity events, or to put it another way, activity features characterizing a first user activity event that are similar to activity features characterizing a second user activity event. The activity features may include features relating to contextual information and features determined by semantic information analyzer 262. Activity events having in-common activity features may be used to identify an activity pattern, which may be determined using activity pattern determiner 266.

For example, in some embodiments, features similarity identifier 264 may be used in conjunction with one or more pattern-based predictors 267 (a sub-component of activity pattern determiner 266) to determine a set of user activity events that have in-common features. In some embodiments, this set of user activity events may be used as inputs to a pattern-based predictor, as described below. In some embodiments, features similarity identifier 264 comprises functionality for determining similarity of periodic-based and behavioral-based activity features. Periodic-based features comprise, for example, features that may occur periodically. For example, this may include features that occur on a day of the week or month, even/odd days (or weeks), monthly, yearly, every other day, every 3^{rd} day, etc. Behavior-based features may comprise behaviors such as user activities that tend to occur with certain locations or activities occurring before or after a given user activity event (or sequence of previous activity events), for example.

In embodiments where activity features have a value, similarity may be determined among different activity features having the same value or approximately the same value, based on the particular feature. For example, a time stamp of a first activity happening at 9:01 AM on Friday and a time stamp of a second activity happening at 9:07 AM on Friday may be determined to have similar or in-common time stamp features.

Activity pattern determiner 266 is generally responsible for determining a user activity pattern based on similarities identified in user activity information. In particular, activity pattern determiner 266 (or activity pattern inference engine 260) may determine a user activity pattern based on repetitions of similar activity features associated with a plurality of observed activity events. Thus, for example, an activity pattern may be determined where activity features corresponding to two or more activity events are similar. In some instances, an activity event may have many corresponding activity features, which may be represented as a feature vector associated with a particular activity event. Accordingly, the analysis carried out by activity pattern determiner 266 may involve comparing the activity features from feature vectors of a plurality of activity events.

In some embodiments, activity patterns may be determined using pattern inferences logic 230. Pattern inferences logic 230 may include rules, associations, conditions, predictions, classification models, and/or pattern inference algorithms. The pattern inferences logic 230 can take many different forms depending on the particular activity pattern or the mechanism used to identify an activity pattern, or identify feature similarity among observed activity events to determine the pattern. For example, some embodiments of pattern inferences logic 230 may employ machine-learning mechanisms to determine feature similarity, or other statistical measures to determine the activity events belonging to a set of “example user actions” that support the determined activity pattern, as further described below. The user activity information may be received from user activity monitor 280 and information about identified similar features may be received from features similarity identifier 264. In some embodiments, the user pattern(s) determined by activity pattern determiner 266 may be stored as inferred activity patterns 248 in user profile 240.

In some embodiments, activity pattern determiner 266 determines or infers a pattern of user activity and an associated confidence score regarding the strength of the user activity pattern. More specifically, in some embodiments, a corresponding confidence weight or confidence score may be determined for an identified user activity pattern. The confidence score may be based on the strength of the pattern, which may be determined based on the number of observations (of a particular user activity event) used to determine the pattern, how frequently the user's actions are consistent with the pattern, the age or freshness of the activity observations, the number of similar features, types of features, and/or degree of similarity of the features in common with the activity observations that make up the pattern, or similar measurements. In some aspects, the confidence score may be provided with the determined activity pattern to an optimal schedule(s) generator 290 for use in generating one or more optimal schedules. For example, regarding the optimal schedule generator 290, in some embodiments, a minimum confidence score may be needed before a user activity pattern is determined and/or utilized for generating an optimal schedule and/or otherwise used to provide an improved user experience, such as with optimal-schedule consumer application(s) 270.

Some embodiments of activity pattern determiner 266 determine a pattern according to the example processes described below, where each instance of a user activity event has corresponding historical values of tracked activity features (variables) that form patterns, and where activity pattern determiner 266 may evaluate the distribution of the tracked variables for patterns. In the following example, a tracked variable for a user activity event is a time stamp corresponding to an observed instance of the user activity event. However, it will be appreciated that, conceptually, the following can be applied to different types of historical values for tracked activity features (variables).

A set of time stamps (i.e., values of a given tracked variable) can be denoted as {tₘ}ₘ₌₁^{M}, and mapped to a two-dimensional histogram of hours and days of the week. The two-dimensional histogram can comprise a summation over the instances of the user-device interaction, such as:

hᵢⱼ=Σₘ₌₁^{M}I[dayOfWeek[tₘ]=i]∧I[hourOfDay[tₘ]=j].

This histogram can be used to determine derivative histograms. For example, a day of the week histogram may correspond to: hⱼ=Σᵢhᵢⱼ. An hour of the day histogram may correspond to: hᵢ=Σⱼhᵢⱼ. As further examples, one or more histograms may be determined for particular semantic time resolutions in the form of: h_{iC}=Σ_{jϵC}hᵢⱼ. Any of various semantic time resolutions may be employed, such as weekdays and weekends, or morning, afternoon, and night. An example of the latter is where Cϵ{morning, afternoon, night}, morning={9, 10, 11}, afternoon={12, 13, 14, 15, 16}, and night={21, 22, 23, 24}.

An additional data structure utilized in representing an event can comprise the number of distinct time stamps in every calendar week that has at least one time stamp therein, which may be represented as:

wᵢ^{j}=∥{m|tₘ is within the i-th j week period}∥.

As an example, w₂³ can denote the number of distinct time stamps during the 2^{nd} three-week period of available time stamps. N^{(j)} may be utilized to denote the number of j-week time stamps available in the tracked data; for example, N⁽³⁾ denotes the number of three-week periods available in the time stamps.

Activity pattern determiner 266 (or activity pattern inference engine 260) may generate a confidence score that quantifies a level of certainty that a particular pattern is formed by the historical values in the tracked variable. In some embodiments, a corresponding confidence weight or confidence score may be determined for the user activity pattern by the activity pattern determiner 266 or the activity pattern inference engine 260. The confidence score may be based on the strength of the pattern, which may be determined by the number of observations used to determine the pattern, how frequently the user activity is consistent with the pattern, the age or freshness of the activity observations, the number of features in common with the activity observations that make up the pattern, or other similar measurements. In some instances, the confidence score may be utilized when generating an optimal schedule. For instance, stronger patterns may be more influential (i.e., a generated optimal schedule may be more likely to include an allocation of time for a user activity or activity type indicated by a strong activity pattern). For example, an optimal schedule may include time for eating lunch at noon each day where the user's pattern indicates the user nearly always eats lunch at that time. Further, in some embodiments, a minimum confidence score may be needed before using the activity pattern to generate an optimal schedule. For example, in one embodiment, a threshold of 0.6 (or just over fifty percent) is utilized such that only activity patterns having a 0.6 (or greater) likelihood of predicting user activity may be considered. Other thresholds may be used. In any case, when confidence scores and thresholds are used, determined patterns of user activity with confidence scores less than the threshold may still be monitored since additional observations of user activities may increase the confidence for a particular pattern.

In the following example, the above principles are applied utilizing Bayesian statistics. In some implementations, a confidence score can be generated for a corresponding tracked variable that is indexed by a temporal interval of varying resolution. For time stamps, examples include Tuesday at 9 AM, a weekday morning, and a Wednesday afternoon. The confidence score may be computed by applying a Dirchlet-multinomial model and computing the posterior predictive distribution of each period histogram. In doing so, a prediction for each bin in a particular histogram may be given by:

$${x}_{i} = \frac{{α}_{0} + {h}_{i}}{{\sum }_{i}^{K} ( {α}_{0} + {h}_{i} )} ;$$

where K denotes the number of bins, α₀ is a parameter encoding the strength of prior knowledge, and

$$\frac{10 + 4}{( 10 + 3 ) + ( 10 + 4 ) + ( 10 + 3 )} = \frac{14}{40} \approx 0.35 .$$

Then, the pattern prediction is the bin of the histogram corresponding to i* and its confidence is given by xᵢ*. As an example, in a histogram in which morning=3, afternoon=4, and evening=3. Using α₀=10, the pattern prediction is afternoon, and the confidence score is

$${i}^{*} = arg {max}_{i} {x}_{i} .$$

In accordance with various implementations, more observations result in an increased confidence score, indicating an increased confidence in the prediction. As an example, consider a histogram in which morning=3000, afternoon=4000, and evening=3000. Using a similar calculation, the confidence score is

$$\frac{4010}{10030} \approx 0.4 .$$

Also, in some implementations, a confidence score can be generated for a corresponding tracked variable that is indexed by a period and a number of time stamps. Examples include one visit per week, and three visits every two weeks. Using a Gaussian posterior, a confidence score may be generated for a pattern for every period resolution, denoted as j. This may be accomplished by employing the formula:

$$= λ ( \frac{1}{{N}^{( j )}} {\sum }_{i}^{{N}^{( j )}} {w}_{i}^{( j )} ) + ( 1 - λ ) {μ}_{0} , where λ = \frac{{σ}_{0}^{2}}{\frac{{σ}^{2}}{{N}^{( j )}} + {σ}_{0}^{2}} .$$

In the foregoing, σ² is the sample variance, and σ₀² and μ₀ are parameters to the formula. A confidence score can be computed by taking a fixed interval around the number of time stamps prediction and computing the cumulative density as:

$${conf}_{j} = P ( \vert x - \vert < a ) = {\int }_{- a}^{+ a} 𝒩 ( x \vert , {\hat{σ}}^{( j )} ) , \\ where {\hat{σ}}^{( j )} = \frac{1}{\frac{{N}^{( j )}}{{σ}^{2}} + \frac{1}{{σ}_{0}^{2}}} .$$

As an example, consider the following observations: w₁⁽¹⁾=10, w₂⁽¹⁾=1, w₃⁽¹⁾=10, w₄⁽¹⁾=0, w₁⁽²⁾=11, and w₂⁽²⁾=10. N⁽¹⁾=4 and N⁽²⁾=2. Using μ₀=1 and σ₀²=10, μ⁽¹⁾=4.075, and conf₁=0.25. Furthermore, μ⁽²⁾=10.31 and conf₂=0.99. In the foregoing example, although fewer time stamps are available for two week periods, the reduced variance in the user signals results in an increased confidence that a pattern exists.

Having determined that a pattern exists, or that the confidence score for a pattern is sufficiently high (e.g., satisfies a threshold value), activity pattern determiner 266 may identify that a plurality of user activities corresponds to a user activity pattern for the user. As a further example, activity pattern determiner 266 may determine that a user activity pattern is likely to be followed by a user when one or more of the confidence scores for one or more tracked variables satisfy a threshold value.

In some embodiments, patterns of user activity may be determined by monitoring one or more activity features, as described previously. These monitored activity features may be determined from the user data described previously as tracked variables or as described in connection with user-data collection component 210. In some cases, the variables can represent context similarities and/or semantic similarities among multiple user actions (activity events). In this way, patterns may be identified by detecting variables or features in common over multiple user actions. More specifically, features associated with a first user action may be correlated with features of a second user action to determine a likely pattern. An identified feature pattern may become stronger (i.e., more likely or more predictable) the more often the user activity observations that make up the pattern are repeated. Similarly, specific features can become more strongly associated with a user activity pattern as they are repeated.

In some embodiments, such as the example embodiment shown in system 200, activity pattern determiner 266 includes one or more pattern-based predictors 267. Pattern-based predictors 267 comprise one or more predictors for predicting a next or future user action likely to be taken by the user based on activity patterns, such as patterns of behavior or similarity features. At a high level, a pattern-based predictor 267 receives user activity information and/or activity features associated with a user activity and determines a prediction of the next action or a future action to be taken by the user. This prediction may, for example, be directly used by the optimal schedule(s) generator 290 to determine which future user activities to include in an optimal schedule. In an embodiment, the pattern-based predictor 267 includes functionality, as further described below, for performing user activity filtering, determining an activity score, selecting an activity or activity type based on the score, and determining a particular pattern-based prediction, which can be provided to the optimal schedule generator 290 for use in generating an optimal activity schedule that includes a future activity or activity type corresponding to the pattern-based prediction.

In one embodiment, a pattern-based predictor 267 uses features similarity identifier 264 to determine features or patterns in common between historical user activities and a recent user activity. For example, similarity of periodic features may be determined from among the set of historical user actions and/or from those historical actions having a periodic feature in common with a current or recent user action. Thus, for example, if a recent user action happened on a Monday, on the first day of the month, on an even week, or on a weekday, then determining periodic features similarity would comprise identifying those historical user actions that have features indicating the user action happened on a Monday, those historical user actions having features corresponding to a first day of the month (any first day, not just Mondays), or happening on an even week, or a weekday. Likewise, behavior features similarity may be determined in order to identify sets of historical user actions having a particular behavior feature in common with a current or recent user action. For example, if a recent user action includes opening a certain type of content (e.g., a word document), then determining behavior features similarity would comprise identifying those historical user actions that have features indicating the user opened the same type of content.

User activity filtering may use the feature similarity determinations to filter out historical user actions and retain only those historical user actions that have a particular feature (or features) in common with a current or recent user action. Thus, in some embodiments, each pattern-based predictor 267 may be designed (or tuned) for determining a prediction based on a particular feature (or features). For example, there might be a subset of pattern-based predictors 267 used for determining a prediction when the feature indicates a workday, or weekend, or Monday, or particular application access, application usage duration or another indication. Such a pattern-based predictor 267 may need only those historical user actions corresponding to its prediction model. In some embodiments, such pattern-based predictors may utilize specific prediction algorithms or models, based on their type of pattern prediction (prediction model). These algorithms or models may be stored with pattern inferences logic 230 in storage 225.

Accordingly, in some embodiments, for each pattern-based predictor 267, user activity filtering may be carried out to determine a set of historical user actions that are relevant to that particular pattern-based predictor 267, which may include, for example, periodic-feature-based predictors, behavior-feature-based predictors (which may include behavior sequences or sequences of previous user actions), unique or uncommon behavior features (such as when a user performs an activity at an unusual time when compared to similar historical user activities) or other types of feature-based predictors. In some embodiments, features similarity identifier 264 may determine user activity sequence similarity (e.g., the sequence of the last K user actions prior to the current action (or a particular recent action) by determining a Levenschtein distance between the historical user action sequence and recent user action sequences).

Additionally, in embodiments where user activity filtering may be performed such that each predictor 267 determines a subset of historical user actions with features that correspond to its prediction criteria, for each predictor 267, for the subset of historical user actions that pass the filter, user action scoring may be performed on the subset of features. User action scoring generally compares similarities of features in the current or recent user action and the subset of historical user actions (which may be considered as a comparison of contexts) and scores each user action with respect to the similarity of its features. In particular, some embodiments score not only those features used for determining the subset of historical user actions, but all (or a larger number) of features available in the current or recent action and historical actions for comparison. In some embodiments, a Boolean logic process is used (i.e., the features have to be true or have the same pattern, and if this is satisfied, then the difference between the particular features is determined). The differences may include, for example, differences in the time-related features, usage duration, sequence distances, or other differences. In an embodiment, these differences are determined and put into a sigmoid. Further, in an embodiment, a similarity threshold is used, which may be pre-determined, tunable, or adaptive, or may be initially set to a value based on a population of users or may be based on empirical information learned about the particular user, for example, and may be adaptive based on the number of historical observations. The similarity threshold may be used to determine whether a particular historical user action is “similar enough” to the particular current or recent user action so as to be considered for determining a prediction. In some embodiments, a vector representing the similarity differences (or similarity score) may be determined, for example, where multiple features are evaluated for similarity.

The user actions from the subset of historical user actions that are most similar (or similar enough) to the particular current or recent user action may be selected. In some embodiments, the selection process uses a similarity threshold, such as described above, to determine those historical user actions that satisfy the similarity threshold. It should be noted that although the term “selection” is used, it is contemplated that the selection is performed by a computer-related process that does not require a person to perform the selection. The selected historical user actions comprise a set of “example user actions.”

In some embodiments, a prediction of the user's next action (or future action) may be inferred based on (or according to) the historical user actions in the set of example user actions. In an embodiment, the predicted future action is the next or future user action with the highest observations count (i.e., the next user action that is predicted the most based on the set of example user actions). Those historical user actions in the set of example user actions that are consistent with the prediction comprise a “prediction support set.” In some embodiments, a prediction probability corresponding to the prediction may be determined, for example, based on a ratio of the size of the prediction support set versus the total number of observations (historical user actions in the subset determined by the user action filtering). Moreover, in some embodiments, the prediction may also comprise additional information related to the predicted user action(s), such as activity features which characterize the predicted action(s). By way of example and not limitation, if the predicted action is that the user will email a work colleague (e.g., which may be determined based on a requested commitment, such as an email from the work colleague that includes “please confirm receipt” or “please let me know . . . ” for example), additional activity features may indicate the specific email recipient, the subject matter of the email, the approximate time the user will access his email program to compose the email, or other information related to the predicted action. The related information may also be determined based on the activity features of the prediction support set observations. Further, the predicted future activity may be provided to the optimal schedule generator 290 so that it can generate an optimal schedule that includes a scheduled time for sending the email consistent with the expected future time, and also, in some embodiments, may associate the relevant information with the optimal schedule, such as the email recipient, the subject matter, or other relevant information.

Some embodiments also determine a prediction significance, which may be determined based on a confidence interval (e.g., a Binomial confidence interval) or other appropriate statistical measure. Still further, in some embodiments, the prediction confidence may be based on the prediction probability and the prediction significance (e.g., the product of the prediction probability and the prediction significance). Accordingly, some embodiments of activity pattern determiner 266 provide a predicted future user activity (or type of activity) or series of future activities or types of activities for each of the pattern-based predictors 267.

Some embodiments determine a specific prediction from the one or more pattern-based predictors 267. In an embodiment, an ensemble process is utilized wherein the one or more pattern-based predictors 267 vote, and a selection is determined based on the ensemble-member predictors. Further, in some embodiments, ensemble-member predictors may be weighted based on learned information about the user actions. In one embodiment, once each pattern-based predictor 267 has provided a prediction, the prediction that has the highest corresponding confidence is determined as the future user activity, and may be considered a pattern-based (or history-based) prediction. In some embodiments, the output of activity pattern determiner 266 may be stored as inferred activity patterns 248 in user profile 240 or provided to optimal schedule generator 290.

Continuing with the system 200 depicted in FIG. 2, the optimal schedule generator 290 is generally responsible for generating one or more optimal schedules of user activity for a user. In some embodiments, optimal schedule generator 290 is configured to receive user data (e.g., from user-data collection component 210 and/or user profile 240), user activity information associated with the user and/or the user's computing devices and/or applications (e.g., from user activity monitor 280 and/or user activity information 242 in user profile 240), and the determined user activity patterns (e.g., from activity pattern inference engine 260 and/or inferred activity patterns 248 in user profile 240). The user data and user activity information may include historical, real-time, and/or near real-time information. Optimal schedule generator 290 may then utilize this information to generate one or more optimal schedules of user activity for a particular user. In some embodiments, the generated optimal schedule may be stored in optimal schedules 249 of user profile 240.

In some embodiments, optimal schedule generator 290 also determines and/or allocates future activity likely to be performed by the user. For example, future activity may be determined based on explicit or inferred future activity or future types of user activity. Inferred future activity (or types of activity) may be determined from user activity patterns (and may be provided by activity pattern inference engine 260 or pattern-based predictor 267, as described above) or from user data indicating likely future activity, such as communications about an upcoming meeting or an upcoming project deadline, for example, where the likely meeting time(s) may be inferred from the communications (e.g., based on a person including a requested meeting time in a communication). In some instances, future activity may be inferred based on past or historic user activity performed in response to, or in association with, similar user data. For example, user activity performed when the user encountered another project deadline in the past may be used to infer future activity for the current project deadline.

Further, user activity patterns may be used to infer future activity (or types of activity). For example, a user activity pattern that indicates the user typically spends 1-2 hours throughout the day responding to emails may be used to infer that future activity performed by the user will likely include responding to emails. Thus, an optimal schedule generated by optimal schedule generator 290 may need to include time for responding to emails. But, a particular optimal schedule generated by optimal schedule generator 290 may allocate the “responding to emails” activity only to certain time(s) of the day that are optimal for the user's workflow. Similarly, where a user's activity pattern indicates that the user works out three days per week for one hour, then it may be inferred that future activity of the user will likely need to include time for working out. But again, a particular optimal schedule may allocate working out to a certain time of the day or days of the week that are in harmony with the user's natural behavior patterns. Future activity may also be inferred from explicit information, such as information derived from a user's calendar(s), project timelines or work-flows, task lists, assignments, or other user information indicating explicit future activities to be performed. Other future user activities may be determined as described elsewhere herein.

In some embodiments, an optimal schedule includes an allocation of time specifying a future user activity or type of future user activity. For example, an optimal schedule may include allocations of time for types of user activities such as focus-intensive work, non-focus-intensive work, multitasking, recreational activity, traveling, sleeping, meetings/collaborative work, communicating or responding to communications, planning, organizing and preparing, reading, writing, coding, programming, calculating, analyzing, creating or editing content, or other types of user activity.

Each optimal schedule may further be generated by the optimal schedule generator 290 based on determined context associated with the user. Such context may be a location of the user, timing of user activities, application and/or computing device activity and usage, user communications and content thereof, and/or other contextual information. For example, one or more optimal schedules may be generated for a user that include allocations (or blocks of time) for meeting with a collaborator on a project. It may be determined from contextual information, such as the user's communications (e.g., content of an email), that the user will be traveling for several days, and will not be available to meet with the collaborator during the period of travel. This contextual information may be used to modify or generate a new optimal schedule that suggests collaborative meetings only when the user and the other person are actually able to meet (e.g., before or after the user travels).

As described herein, in some embodiments, optimal schedule generator 290 generates a plurality of optimal schedules. The different optimal schedules may be generated based on a future probability of a user performing different user routines and/or contexts; for example, where user activity patterns indicate a user has a sixty-percent chance of traveling and a forty-percent chance of not traveling, then two (or more) optimal schedules may be generated to correspond to each scenario. Thus, in some instances, optimal schedule generator 290 may generate multiple, mutually exclusive schedules, and the current context of the user (e.g., determined based on contextual information) may be utilized (by optimal schedule generator 290 or by one or more optimal-schedule consumer applications 270) to select an appropriate optimal schedule. For example, where context indicates the user is traveling and out of the office, then an optimal schedule that includes allocations of time for future activities related to the user's travel (e.g., the purpose of the travel, such as preparing for a presentation or an in-person meeting) may be used instead of an optimal schedule that includes allocations of time for the user to attend office meetings at the user's work place.

Similarly, multiple optimal schedules may be generated based on different patterns of user activity. For example, one optimal activity schedule may be generated based on a first pattern of user activity (e.g., a normal workday for the user at a normal work location) and another optimal activity schedule may be generated based on a second pattern of user activity (e.g., a day on which the user is traveling or working at home). As further described herein, user context and/or other user data, which may include feedback or user preferences, may be utilized to determine a most probable or appropriate optimal schedule (such as the optimal schedule that most corresponds to the user's circumstance), which then may be provided to an optimal-schedule consumer application 270 or otherwise incorporated into the user's computing applications and services (such as adding a suggested user activity to the user's calendar at the designated time).

Furthermore, in some embodiments, an optimal schedule may be updated as user data indicates new information. For example, after an optimal schedule has been utilized by an optimal-schedule consumer application 270 and after a user's schedule has progressively advanced, one or more additional optimal schedules may be generated based on real-time changes of the user's activity, which may indicate that other user activity patterns are likely being carried out. For example, if a user deviates from a planned set of activities (e.g., due to unanticipated meetings, travel, or personal needs), an additional optimal activity schedule may be generated and utilized. In particular, it may be determined that the deviated patterns of activity correspond to a secondary, known pattern of activity that the user sometimes engages in, such as traveling to a work-related remote location for a business meeting. In this case, one or more updated optimal activity schedules corresponding to the secondary pattern of activity may be generated and utilized by an optimal-schedule consumer application 270 (e.g., so that user activities that are presented to the user correspond to this secondary pattern of activity; for example, the optimal schedule may include allocations of time for travel activities corresponding to when the user is expected to be traveling to the remote location).

Further, an optimal activity schedule may be generated that allocates likely future user activities or activity types according to the best available options. For example, the future activities may be allocated based on determined changes in user activity (e.g., the user is transitioning to a new location), user commitments (e.g., a new deadline for a project), predicted future user activity (e.g., scheduling several hours of work for the project), and other current context (e.g., time, location, associated persons, application and computing device usage, communications, or other context). As a further example, an optimal activity schedule may allocate future user activities to periods of time that match a user's context. If a user is traveling and needs to perform focus-intensive, user-only activity (e.g., reading, writing, programming, coding, planning, or other similar activities), and current context indicates that the user will be on an airplane flight for three hours with minimal outside connectivity, the optimal schedule may schedule the focus-intensive activity during the airplane flight to match the future activity with a correspondingly appropriate time. Unless, for instance, the user has indicated in user preferences not to allocate work during travel.

User feedback may also be used to generate an optimal schedule for a user. User feedback may be, for example, explicit feedback, such as input from a user indicating a desire to schedule a certain activity during a certain time period (e.g., a user-created task list indicating the user wants to schedule work on a certain day), or implicit feedback, such as user actions that indicate a preference (e.g., initiating or closing an application at certain times, modifying content at certain times, accepting or declining recommendations for particular user activities, performing particular activities at certain times, or other implicit feedback). For example, a user may indicate a preference by setting a reminder to “work on my manuscript for two hours this week,” which may be used to determine a desired future user activity (i.e., writing and editing the user's manuscript) and a desired future time period (i.e., a period of time in the forthcoming week). Patterns of user activity may indicate that the user often does this type of activity (e.g., activity categorized as reading/writing, work-related activity, user-only activity, or this exact activity) during the morning on weekdays. An optimal schedule may therefore be generated by optimal schedules generator 290 with this activity scheduled for at least one morning in the forthcoming week during a time interval that the user typically performs such activity. In this sense, a generated optimal schedule may incorporate user-indicated preferences, needs, obligations, and/or commitments.

In some embodiments, a user feedback component 250, discussed further below, may collect from a user or enable the user to provide explicit feedback regarding an optimal schedule utilized by one or more optimal-schedule consumer applications, such as optimal-schedule consumer applications 270. For example, in one embodiment, a user may be able to access and review the optimal schedule(s) generated by optimal schedule generator 290 and to modify the schedule(s) or provide feedback, providing the user an opportunity to provide user feedback and/or preferences that then may be used by the optimal schedule generator 290.

A user may also specify explicit preferences or settings (which may be specified in user preferences 246 of user profile 240) regarding specific allocations of time for optimal schedules. In some embodiments, such settings may be specified as user requirements or desires; for instance, the user may specify that at least one hour of the user's optimal schedule must include an allocation for focus-intensive work. Or, the user may specify that the user desires or prefers to have at least one hour of focus-intensive work. In one embodiment, a graphical user interface for receiving user preferences may include a slider corresponding to the degree of user desire for the particular preference, further enabling the user to tune their optimal schedule(s). Additional details of user preferences for optimal schedules are described in connection with user preferences 246.

A user-generated task list, such as an application-based task list stored on the user's computing device(s) and/or on the cloud (e.g., in a user's Microsoft Outlook account) may be used to determine specific future activities to include in an optimal activity schedule (e.g., a user has a task list that includes the following user-generated tasks: go to grocery store, go to watch repair store, go to daughter's recital, and meet family for dinner). In some embodiments, tasks may be inferred from the task list. For instance, for the task “buy milk”, it may be inferred that the future activity will comprise going to the grocery store or more generally, shopping. In some embodiments, optimal schedule generator 290 may consider user data from a user's calendar such that the predicted future activities may be cross-referenced with available times in the user's calendar to match the user's availability with predicted future activities.

In some embodiments, user activity information included in an optimal schedule may also include information for designations for an availability, user status, or presence indicator associated with the user for each of the activities (e.g., each activity may have associated therewith a status indicator designation, such as available, busy, or inactive). Such information may be utilized by an optimal-schedule consumer application, such as consumer application 273, to provide notifications or indications to other users of the user's availability, further facilitating and streamlining communication and interactions with other users (e.g., other users may see the availability, presence, or status indicator on the user's schedule or communication applications). This may facilitate interactions with other persons at appropriate times of availability. In some embodiments, an availability, presence, or status indicator may be modifiable by the user and/or visible to the user and/or other persons, and/or may be automatically modified in response to activities or events being integrated into the user's schedule. For example, when a user schedules a meeting for an hour, and the presence indicator indicates “busy” for that hour, the same presence indicator status may also be applied to the 30 minute period before the meeting, based on activity patterns indicating the user usually prepares for meetings for this amount of time.

In some embodiments, predicted future user activity information included in an optimal activity schedule may be determined from the content of user-related communications (e.g., emails, text or instant messages, voice messages, video and audio recordings, Snapchat® communications, Slack® communications, Skype® communications, Jabber® communications, etc.). In this regard, some embodiments of optimal schedule generator 290 may include one or more analytical applications or services configured to review the content of user communications (both user-initiated and communications received from other persons) to designate communications and content thereof that include probable or likely future user activities that should be included in an optimal activity schedule. These analytical applications or services may utilize topic modeling or automatic summarization (which may include extraction or abstraction) to analyze the content and extract information indicating action items or similar user activity from the content of the communications. In some embodiments, a weighting factor may be applied based on the topic, the persons involved, the level of relation to the user, a frequency of occurrence of the subject in communications, and/or other factors. For example, a user may send an email stating “let's get together to discuss the proposal this week.” Based on the content of the communication indicating a meeting in the next week, and in view of the user's schedule and location, the communication participants' schedules and locations, the user's activity patterns related to meetings and work related to the proposal, and/or indicated or determined user activity preferences (e.g., times at which the user prefers scheduling meetings so that they do not interfere with other work or activities), an optimal schedule may be generated in which a proposed meeting to discuss the proposal is included. The optimal schedule may be utilized by an optimal-schedule consumer application, such as consumer application 271 discussed further below, to provide a calendar invite for the meeting that may be automatically generated and/or sent to all participants.

In this example, the analytical application or service that reviews the content of the user's communications may automatically and/or continuously review user communications to determine potential future user activities for assessment of their inclusion into the user's optimal activity schedules. The analytical application or service may also rank predicted future user activities extracted from the communications based on different factors, such as importance, relevance, and/or consistency with other user activity events and patterns. For example, potential ranking factors may be participants (e.g., a user's boss may be considered more important than a co-worker), content (e.g., a topic, and whether the topic is new or frequently engaged with by the user), and/or user-indicated preferences (e.g., a rule indicating any communications related to “Project ABC” be automatically filtered for scheduling to help the user manage the frequent needs of “Project ABC” more effectively).

In some embodiments, determining user activity information to include in an optimal schedule may further be determined based on a user's calendar events and/or calendar history. For example, the proximity a user schedules certain types of activity events in her calendar to other activity events (e.g., scheduling back-to-back meetings or events vs. spacing out events), including at what times and/or time intervals, may be used to determine or predict future user activity. Activity patterns may also be used to determine things such as duration of user activities included in an optimal schedule generated for a user. For example, it may be determined, such as from past calendar events, that a user prefers meetings to last no more than one hour (e.g., based on calendar scheduling, and/or based on contextual indicators, such as a change in user location which likely indicates a meeting has ended).

Further, an optimal schedule may be generated based on an identification of competing calendar events, taking into consideration a degree of importance of certain calendar events. In this sense, scheduling events for the same time period may be avoided to prevent double-booking of the user. Further, in the case of a conflict, a prioritization of the predicted future activity and any scheduled user activity may be performed to determine which activity has a higher priority (e.g., this determination may be based on historic patterns of user activity indicating which activity is more frequently performed without deviation, or is moved or rescheduled less, or is indicated to be important by the user). For example, a user may have a calendar event indicating “buy concert tickets,” which may be determined to have a lower priority than “work meeting,” and therefore an optimal schedule may suggest scheduling the “work meeting” at the specified time, and scheduling “buy concert tickets” for another time.

User preferences, which may be either explicit or implicit as discussed herein, and patterns of user activity may be used for scheduling, rescheduling, and/or cancelling certain user activities when generating an optimal schedule. These user preferences may be received by optimal schedule generator 290 from user preferences 246 of user profile 240. For example, a certain type of scheduled user activity, such as a weekly meeting, may have a pattern of being frequently (i.e., with greater regularity than certain other events) moved or cancelled, indicating a relatively lower level of importance or significance to the user. This pattern of user activity may be incorporated into an optimal activity schedule for use in selecting or prioritizing user activities. For example, when generating an optimal schedule that faces conflicting scheduling, a pattern of cancelling or changing certain activities may be used to determine that those certain activities can be rescheduled or cancelled to favor other activities that have a higher prioritization (e.g., based on more participants, a certain type of participant (e.g., a user's boss), a topic (e.g., an project with a deadline), a pattern of being less frequently cancelled, or based on another reason). This may include automatically sending an update to participants of a cancelled or rescheduled calendar event or user activity. Similarly, a user activity with a relatively higher determined level of importance may be assigned, in an optimal schedule, to a time period where user activity patterns indicate the user activity is more likely to occur without interruption, cancellation, or rescheduling, or at a time at which the user prefers or regularly schedules user activities with a relatively higher determined level of importance.

The optimal schedule generator 290 may further schedule user activities taking into consideration user activity patterns, current context, and/or user feedback when determining a duration and time for the activities in the optimal schedule. For example, it may be determined that a user needs to schedule a meeting with another person (e.g., based on communication context indicating such a need). In one circumstance, activity patterns of the user determined or inferred by the activity pattern inference engine 260 may indicate that the user typically schedules meetings to last one hour, and typically in the afternoon. In another circumstance, a context extracted from a user communication by the user activity monitor 280 indicates the user has a preferred time and duration for the meeting (e.g., the following day at 3-4 PM). In another circumstance, user-indicated feedback from user preferences 246 in user profile 240 or from feedback component 250 indicates that the user prefers the following day at 3-4 PM for the meeting. Based on any one or combination of such indicators, an optimal schedule may be generated that includes a meeting activity scheduled at 3 PM the next day for one hour. In some embodiments, the optimal schedule generator 290 may generate alternative schedules with longer or shorter durations for the user activity, different scheduled times for the user activity, or alternatives based on availability of other persons involved in the user activity, for example, to provide different options that facilitate efficient scheduling of the meeting (e.g., these alternatives may be provided if the user or another person declines the scheduled activity that is provided). Such alternative schedules may be generated in some embodiments based on indicated availability of the user or other persons, or based on a user declining the suggested time and/or duration of the user activity.

Additionally, contextual information may be used to determine potential user commitments that are likely to become future user activity that is then included in an optimal schedule (e.g., for suggestion as a calendar item). For example, such contextual information may be obtained (by using contextual information extractor 284) from user communications (e.g., email, text messages, voice messages, or other communications), which may be analyzed for potential user commitments and their related information, as described herein. For example, a user may indicate in an email to another person that the user will respond, provide content, schedule a meeting, or perform another activity. This indicated future user activity may be identified as a potential commitment. Accordingly, optimal schedules generator 290 may provide one or more optimal schedules that include scheduling of this potential commitment. Contextual information that may indicate potential commitments may include meeting invites, feedback requests, confirmations of requests, revisions of content or approving of content, email requests, text requests, voice messages, or other indications, notifications, or communications to or from persons confirming or suggesting a user activity be performed. A user's social media interactions, communications, and/or activity may also be analyzed for user commitments to include in an optimal schedule.

In some instances, a user's activity events and activity patterns may be parsed to determine which user activities have occurred in the past and which are likely to occur again in the future. This may be determined, in part, from an analysis of the user's past activities or calendar events, commitments, indicated preferences, and other patterns of user activity. For example, a type of user activity (e.g., work-related, personal-related, activity-related, content-related, or another type of user activity) may be determined, a frequency of occurrence of the user activity (e.g., recurring weekly being more likely to occur again in the future) may be determined, a context of the user activity may be determined (e.g., location of the user, persons the user is interacting with, or other context) and any user preferences (e.g., a user accepting or declining certain scheduled user activities in the past) may be used to tailor or tune the selection of user activities included in an optimal schedule.

In some embodiments, optimal schedule generator 290 may generate different and/or even mutually exclusive optimal schedules (sometimes referred to herein as candidate optimal schedules) for a user based on possible alternative patterns of activity with which the user will be engaged. For example, an optimal schedule may be generated based on the user being engaged in a first recognized pattern of activity (e.g., a pattern corresponding to a normal workday at a normal work location) or based on the user being engaged in a second or alternate pattern of activity (e.g., a pattern corresponding to a non-normal working day, such as one involving travel to a non-typical work location). The separate patterns may be used to generate optimal schedule candidates that can be used to facilitate workflow optimization based on such a distinction. In some instances, and as further described herein, a particular candidate optimal schedule may be selected based on contextual information indicating a user's current context, such as the user traveling or being out of the office. Similarly, in another embodiment, optimal schedules generator 290 can take into account out-of-routine behavior when generating one or more optimal schedules, including based on current context, and/or can also create multiple optimal schedules to account for different patterns of user behavior that might occur as referenced above. For example, if a user books a flight during a workday that will, if not cancelled, necessitate a different user activity schedule than a normal workday (e.g., based on changes in location or availability), the optimal schedule generator 290 may select and arrange user activities in an optimal schedule for that day that take this out-of-routine behavior into account (e.g., choosing activities that are conducive to the user's location, travel plans, and patterns of activity during travel).

An optimal schedule may be generated with user activities (or activity types) allocated based on the user's working habits, so that specific activity that is important to the user and/or activity that requires a higher degree of user focus is scheduled at times that best facilitate optimal performance by the user. For instance, in one embodiment, a generated optimal schedule may allocate focus-intensive or concentration-based activities to periods of time in a user's schedule when the user's cognitive abilities are determined to be the highest. For example, based on the activity patterns of the user indicating that the user has higher productivity, higher engagement with applications or content, and/or higher resistance to distractions (e.g., emails, texts, or other communications) at certain times (e.g., early in the morning between 6 AM and 9 AM), user activities that are focus-intensive may be scheduled for these times by optimal schedule generator 290 to facilitate user productivity. Similarly, activities requiring less focus and concentration may be scheduled for different times that are appropriate for those tasks based on the activity patterns of the user. For example, it may be determined that a user is not highly focused or engaged when the user is surfing the web on their computer during lunch, resulting in an optimal schedule being generated with less focus-intensive activity (e.g., email replies, administrative duties, house-cleaning, or other non-cognitively demanding tasks or short-term tasks) that do not require a high level of engagement (e.g., compared to focus-intensive work like reading, writing, calculating, programming, or creating or modifying content) for this time. In contrast, it may be determined that a user's focus is high first thing in the morning, and user activities requiring cognitive focus may be scheduled during this time.

In some embodiments, optimal schedules may be determined using optimal schedule logic 235. Optimal schedule logic 235 may include rules, associations, conditions, predictions, classification models, and/or scheduling algorithms that utilize user activity, user activity patterns, predicted or future user activity, and/or historical user data to determine aspects of an optimal schedule. In this way, optimal schedule logic 235 enables optimal schedule generator 290 to remove the subjectivity that would otherwise be present in conventional approaches to scheduling optimization, which may be performed by humans. The optimal schedule logic 235 can take many different forms depending on the user activities determined to be likely to occur. For example, some embodiments of optimal schedule logic 235 may employ machine-learning mechanisms or other statistical measures to classify future activity events as types of activity (e.g., a task requiring browsing or light reading may be classified as non-focus-intensive activity) for appropriate allocation in an optimal schedule, or for determining an optimal schedule based on user context, preferences, and/or feedback. In some embodiments, optimal schedule logic 235 may include conditional rules or logic for specific types of future activity, such as logic for automatically including traveling time (and may further include logic to provide an indication that the user is unavailable at that time) when an event, such as a meeting, requires a user to travel to different location. Similarly, where a user behavior pattern indicates the user has a pattern of preparing for meetings or certain types of meetings, optimal schedule logic 235 may include logic for automatically allocating meeting preparation time (and may further include logic to provide an indication that the user is unavailable during this time) if a meeting is scheduled. As yet another example, optimal schedule logic 235 may include logic based on user preferences (or user activity patterns) to avoid scheduling meetings back-to-back, if a user prefers not to have meetings scheduled this way or has a pattern of avoiding scheduling meetings back-to-back. (Similarly, optimal schedule logic 235 may include logic for attempting to schedule meetings at back-to-back times, if user preferences or activity patterns support or prefer this scheduling.)

Continuing with system 200, feedback component 250 is generally responsible for receiving feedback from a user regarding generated optimal schedules, which may be provided to the user through one or more optimal-schedule consumer applications 270. In some embodiments, feedback component 250 comprises a user interface and may be used to solicit, request, and/or provide feedback from a user. For example, based on one optimal user activity schedule that is generated by the optimal schedule generator 290 and recommended to the user, feedback component 250 may generate a user interface element requesting user input indicating an acceptance or rejection of the optimal schedule, or an acceptance or rejection of some of the suggested user activities and/or times of the suggested user activities in the optimal schedule. In some embodiments, a user may provide input that an optimal user activity schedule (or at least a portion thereof, such as one particular scheduled user activity) is accepted, initiating integration of the accepted user activities into the user's calendar, schedule, or other user-related applications and devices, or may decline, in which case no integration occurs and/or a new optimal schedule is generated. For instance, in one embodiment, user feedback may be solicited with a prompt asking if the user would like more or less time for a particular user activity allocated in the optimal schedule. In another embodiment, an allocation of time on an optimal schedule may include a user-interface element, such as a slider or plus-minus enabling the user to increase or decrease the allocated time for that particular activity or activity type.

Further, in other embodiments, the user may indicate, through feedback component 250 (or user preferences 246, described below), how user activities should be scheduled. For example, a user may indicate via feedback component 250 (or user preferences 246) that a user activity should or should not be scheduled at a certain time (e.g., a time, or time interval, or one or more time periods during a time interval, etc.), how often the user activity should occur (e.g., once, daily, weekly, etc.), that a particular user activity has a higher level of importance than other user activities, meaning the important user activity is given priority in scheduling, and/or that a user prefers having a user activity scheduled in conjunction with other user activities (e.g., batching meetings together in sequence). It should further be noted that implied feedback from a user may be used to determine patterns and preferences of the user. For example, when an optimal-schedule consumer application utilizes an optimal schedule, such as by managing the user's calendar or generating a recommended daily schedule for the user, but the user does not accept or follow the calendar scheduling or aspects of the daily schedule, then implied feedback may be determined indicating that the user does not accept the particular optimal schedule that was utilized.

Optimal-schedule consumer applications 270, including applications 271-273, may utilize one or more of the optimal schedules generated by the optimal schedule generator 290 to provide enhanced user experiences. For example, in one embodiment, a generated optimal schedule may be used to provide a suggestion or recommendation to the user to perform a certain activity at a certain time, and/or may be used to provide a request to schedule a recommended user activity at a certain time (e.g., on a user's calendar). (This example functionality may be provided using one or more of the optimal-schedule consumer applications 270.) Optimal-schedule consumer applications 270 may comprise computerized personal assistant applications or services, which may reside on one or more user devices and/or in the cloud. Optimal-schedule consumer applications 270 may receive one or more optimal schedules from the optimal schedule generator 290, and may also receive information about current user activity, which may include a user's current context, from user-data collection component 210, user profile 240, and/or from user activity monitor 280. Three example consumer applications, which provide the enhanced experiences and which are now described, include a calendar management program, which utilizes an optimal schedule for managing a user's calendar events to be more in accordance with the optimal schedule, a personal performance optimizer application, which utilizes an optimal schedule to provide the user with a recommended daily schedule of user activity based on future activity to be performed by the user, and a notifications service, which utilizes an optimal schedule to manage electronic notifications to the user or to other people.

A first optimal-schedule consumer application 270 comprises a computer-performed calendar management program or service 271 that utilizes an optimal schedule for managing a user's calendar events to be more in accordance with the optimal schedule. Some embodiments of program 271 may be integrated into a user's existing calendar or communications application, such as Outlook®, Google® Calendar, or iCloud® calendar. Calendar management program 271 receives, and in some cases, may select (e.g., based on a current context of the user where multiple candidate optimal schedules are provided) an optimal schedule for the user. The optimal schedule may be received from optimal schedule generator 290. Using the optimal schedule, calendar management program 271 then manages a user's calendar or schedule in accordance with the optimal schedule and the activity information included therein. For example, the calendar management program 271 may modify a user's existing calendar to be more consistent with the optimal schedule (such as by rearranging calendar items so that the activity type of the calendar item is consistent with a designated activity type allocated in the optimal schedule). Similarly, calendar management program 271 may manage incoming calendar items, such as new meeting events, so that the scheduling placement of the meeting events is consistent with the optimal schedule. Calendar management program 271 also may automatically generate calendar items based on the optimal schedule, such as creating a calendar event for “focus-intensive work,” thereby making the user unavailable for meetings or other activities to be scheduled at that time. In some embodiments, the calendar management program 271 may incorporate all or portions of an optimal schedule into the user's calendar, schedule, and/or other routine or planning-centered applications (e.g., reminder applications, scheduling applications, social media accounts, a personal digital assistant application, or other scheduling applications or services).

In some embodiments, calendar management program 271 may provide indications of the modifications made to a user's calendar or to other planning or scheduling applications. For example, calendar management program 271 may add or modify a calendar item in the user's calendar based on acceptance of a recommended user activity presented to the user in accordance with a generated optimal schedule. The calendar management program 271 may then provide an indication to the user and/or to others that the user has a scheduled activity or activity type (such as by indicating to other people that the user is busy or unavailable for meetings during the time of the scheduled activity). Further, in some instances the calendar management program 271 may automatically generate communications, such as automatic replies to other person's communications or calendar requests, in accordance with the scheduled calendar items. In some embodiments, the indication may comprise a calendar update indication (e.g., surfacing an alert) or a presence indicator modification (e.g., changing an availability/presence/status indicator in the user's calendar for the scheduled time of the calendar item).

A second optimal-schedule consumer application 270 shown in FIG. 2 comprises a personal performance optimizer computer application 272 that may use one or more optimal schedules to generate a tailored activity schedule that is personalized for the user. For example, in one embodiment, a recommended schedule may be presented to the user in the morning as a suggested daily activity schedule. The recommended schedule may suggest specific user activities or activity types at corresponding times, which are determined by application 272 based on an optimal schedule. In this way, the personal performance optimizer application 272 supports the user's activity habits, preferences, and/or personal attributes, and therefore is likely to improve workflow, time management, and efficiency.

The personal performance optimizer application 272 may receive or select an optimal schedule from the optimal schedules generator 290 or from optimal schedules 249 in user profile 240. In some instances and as described herein, the recommended user activities or activity types may be determined based on inferred user activity determined from one or more user activity patterns. The recommended user activity may also be determined based on explicit user data indicating future tasks, calendar events, or other future activities the user is likely to perform.

More specifically, the personal performance optimizer application 272 may use current user activity and user data (e.g., task lists, calendar items, or inferred future activity) in combination with one or more optimal schedules (which may reflect user behavior patterns) to determine optimal times for a user to perform certain activities or types of activities, such as eating, sleeping, working, commuting, exercising, or other activities described herein. This also may include optimal times for performing different types or categories of activity, such as focus-intensive work, non-focus-intensive work, user-only activity, and/or other categories of activity. These optimal times may be provided in the recommended user schedule and may result in further improving the user's efficiency by reducing the user's time spent planning or scheduling such activities. In some embodiments, the personal performance optimizer application 272 may incorporate current user context into a generated activity schedule for the user, such as a time of day, a user's location, a previous user activity, an upcoming user activity, or computing device and/or application usage, among other contextual information. For example, when a user's location indicates that the user has entered a gym, and when patterns of activity indicate that the user frequently performs cardio workouts at this location, then personal performance optimizer application 272 may utilize an optimal schedule and such context to suggest a cardio workout for the user. Such a recommendation may also be part of a daily schedule provided to the user with the gym activity included.

As a further example, one recommended schedule generated by personal performance optimizer application 272 may include a recommendation for performing focus-intensive work that is frequently performed by a user with limited interruptions at times when the user's ability to remain focused on a cognitively demanding task is determined to be the highest, such as first thing in the morning (if, for instance, user activity patterns indicate that the user is more likely to be focused during this time). In some embodiments, the personal performance optimizer application 272 may recommend a user activity and/or activity schedule based on the user's current context (e.g., the user's location or applications or services being used by the user) so that a suggested user activity is not incompatible with the user's current context (e.g., performing a workout is not recommended while the user is on a plane).

In some embodiments, personal performance optimizer application 272 may include functionality for generating reminders to engage in, switch, or end different user activities. Furthermore, in some embodiments, when generating a recommended schedule, personal performance optimizer application 272 may consider a plurality of optimal schedules received from optimal schedule generator 290, and in some instances may generate a plurality of recommended schedules based on different user routines and/or contexts, allowing a user to select a desired schedule, or allowing one of the plurality of recommended schedules to be used based on an appropriate context or an identified user routine, as described herein.

In some instances, personal performance optimizer application 272 may utilize explicit or implicit feedback from the user when generating a recommended schedule of user activity. For example, user-indicated activities, locations, times, and/or durations, which may be indicated through feedback component 250 or user preferences 246, may be utilized when generating a recommended schedule of activities. The personal performance optimizer application 272 may further make activity recommendations based on commitments of the user, so that recommendations are not incompatible or conflicting with commitments or potential commitments. Personal performance optimizer application 272 may also make suggestions for scheduling certain user activities based on similar users' patterns of activity (such as crowdsourced information from similar users, based on age, gender, work, location, interests, preferences, or other similar factors).

A third optimal-schedule consumer application 270 shown in FIG. 2 comprises a computerized notification service 273 that utilizes an optimal schedule to manage electronic notifications to the user or to other people. The notification service 273 may control the presentation, withholding, and/or modification of electronic notifications to or on behalf of the user based on activity information specified in an optimal schedule. For example, in one embodiment, the notifications service 273 manages communications, alerts, reminders, and/or other notifications that may disrupt the user during her user activity. For instance, when the user is engaged in focus-intensive work in which disruptions are not desirable, the notifications service 273 may reduce, silence, or modify (e.g., delay, reformat to a silent version, or otherwise modify to be less disruptive) notifications or other computer-related events that may cause distraction (e.g., calls, emails, instant messages, alarms, schedule reminders, distracting content which may include advertising, or other notifications) provided or communicated to the user so that the user can remain focused without interruption. The term notifications is used broadly herein and may comprise prompts, alerts, communications, calls, text messages, reminders, other content or other notifications provided to or from the user via a user device, as well as calendar requests, user-feedback requests (e.g., requesting the user confirm or decline a request), or other suggestions that can be viewed, heard, consumed, and/or interacted with by the user. Notifications may include auditory, visual, message-based, and/or graphic-based indicators or alerts, and may be recurring.

In some embodiments, notifications service 273 may be configured to automatically make such adjustments based on scheduled user activities or detected user activities in order to support the user's needs for concentration and focus in such circumstances. For example, when a user is in a period of focus-intensive work, the notifications service may withhold communications from other persons. The communications may be provided to the user in original form under certain circumstances, such as being marked “URGENT.” Otherwise, the notifications service 273 may hold the notifications until the user reaches a period of non-focus-intensive user activity. Additionally, in some instances, rather than withholding the notifications, notifications service 273 may modify the notification or communication, such as by sending or presenting it to the user in another format (e.g., such as a less disruptive format). For example, when someone attempts to call the user, the notifications service 273 may instead present the user with a minimally distracting visual display in the corner of the user's screen, indicating a call is being received. The call sounds may also be silenced so that there is no disrupting sound that interrupts the user. Similarly, a notification for new emails may be modified so that distracting alerts are reduced or eliminated while the user is working. This may be facilitated by batching email alerts and providing them together after a period of time, or by making their display smaller or otherwise less obvious and disrupting. Once the user is engaged in a user activity in which interaction and disruptions are acceptable, the notifications service 273 may again update the electronic notifications to be presented to the user in the normal form.

In some embodiments, the notifications service 273 may be configurable based on user-feedback. For example, in such embodiments, the user may be able to adjust how the notifications service 273 handles electronic notifications directed to the user or to other persons. For instance, a user may adjust a type or size of the notifications presented, an amount of silencing or restriction on the notifications, which types of notifications are allowed or restricted, or other types of adjustments, providing a user's desired management of the notifications. The notifications service 273 may also adjust, based on user activity, an availability, presence, or status indicator associated with the user's applications or services (e.g., adjusting them to indicate that a user is in “do not disturb” status when the user is engaging in focus-intensive work where the user does not want disruptions). The notifications component 273 may utilize the optimal schedule and/or activity information included therein that is integrated into a user's calendar to direct the modification of notifications. The notifications service 273 may also control electronic notifications based on a user's current context (e.g., when a user's activity and/or current context indicates that the user has changed activities and is not engaging in the scheduled focus-intensive activity, and is instead surfing the web, the restriction on notifications may be removed or reduced). The notifications service 273 may also adjust a user's ability to communicate. For example, a user's email or text message application may be restricted, disabled, or hidden when a user is engaged in focus-intensive work according to scheduled user activity from an optimal schedule.

The notification service 273 may also provide time, location, and/or context relevant notifications. For example, the notification service 273 may remind a user which part of a user's scheduled activities the user is engaged in or should be engaged in, including when to switch activities, and may provide updated recommendations to the user suggesting user activities from a generated optimal schedule that correspond to changed user behavior. The notification service 273 may also suggest user activities from an optimal schedule based on the user's current activity or location. For example, a user may schedule focus-intensive work for the first two hours of each working day, and when the user's location indicates that the user has arrived at his or her office, and/or the user's interaction with the user's computing devices and/or applications indicates the same, the notification service 273 may notify the user to engage in this activity, even if the user arrives at a different time each day (e.g., 9:04 AM, 9:15 AM, 9:45 AM, etc.). The notification service 273 may also suggest when a user's optimal time for an activity has arrived or concluded, and may indicate the same to the user with an alert, message, sound, visual display, haptic feedback, or other indication, such as one provided via a user device.

Example system 200 also includes a presentation component 220 that is generally responsible for presenting content and related information to a user. Presentation component 220 may comprise one or more applications or services on a user device, across multiple user devices, or in the cloud. For example, in one embodiment, presentation component 220 manages the presentation of content (e.g., calendar or schedule information, notifications, user interfaces for receiving feedback or user preferences, or other information or content presented to a user, as described herein) to a user across multiple user devices associated with that user. Based on content, logic or rules, device features, and/or other user data, presentation component 220 may determine on which user device(s) content is presented, as well as the context of the presentation, such as how (or in what format and how much content, which can be dependent on the user device or context) it is presented and when it is presented. In particular, in some embodiments, presentation component 220 applies content logic to user device features, optimal schedules, and/or sensed user data to determine aspects of content presentation.

In some embodiments, presentation component 220 generates user interface features associated with the personalized content. Such features can include interface elements (such as graphics buttons, sliders, menus, audio prompts, alerts, alarms, vibrations, pop-up windows, notification-bar or status-bar items, in-app notifications, or other similar features for interfacing with a user), queries, and prompts. In some embodiments, a personal assistant service or application operating in conjunction with presentation component 220 determines when and how to present the content (e.g., withholding or modifying notifications when a user is engaged in focus-intensive work).

Example system 200 also includes storage 225. Storage 225 generally stores information including data, computer instructions (e.g., software program instructions, routines, or services), logic, profiles, and/or models used in embodiments described herein. In an embodiment, storage 225 comprises a data store (or computer data memory). Further, although depicted as a single data store component, storage 225 may be embodied as one or more data stores or may be in the cloud.

As shown in example system 200, storage 225 includes activity pattern inferences logic 230 and optimal schedules logic 235, as described previously, and user profiles 240. One example embodiment of a user profile 240 is illustratively provided in FIG. 2. Example user profile 240 includes information associated with a particular user such as user activity information 242, information about user accounts and devices 244, user preferences 246, inferred activity patterns 248, and optimal schedules 249. The information stored in user profile 240 may be available to the activity pattern inference engine 260, optimal schedule generator 290, or other components of example system 200.

As described previously, user activity information 242 generally includes user information about user actions or activity events, related contextual information, activity features, or other information determined via user activity monitor 280, and may include historical or current user activity information. User accounts and devices 244 generally includes information about user devices accessed, used, or otherwise associated with the user, and/or information related to user accounts associated with the user, for example, online or cloud-based accounts (e.g., email, calendars, task lists or to-do list, social media, or similar cloud-based services with user accounts) such as a Microsoft® account (formerly known as a Net Passport), Google® account, Amazon® account, Wunderlist account, Evernote, other accounts such as entertainment or gaming-related accounts (e.g., Xbox® Live, Netflix®, online game subscription accounts, or other gaming related accounts), user data relating to accounts such as user emails, texts, instant messages, calls, other communications, and other content; social network accounts and data, such as news feeds; online activity; and calendars, appointments, application data, other user accounts, or the like. Some embodiments of user accounts and devices 244 may store information across one or more databases, knowledge graphs, or data structures. As described previously, the information stored in user accounts and devices 244 may be determined from user-data collection component 210 or user activity monitor 280 (including one of its sub-components).

User preferences 246 generally includes user settings or preferences for user activities or activity types that may be included in an optimal activity schedule and/or incorporated from the same into a user's calendar or recommended schedule. In some instances, a user may specify explicit preferences or settings that are stored in user preferences 246 of user profile 240. In some embodiments, such settings may be specified as user requirements or desires; for instance, the user may specify that at least one hour of the user's optimal schedule must include an allocation for focus-intensive work, or the user may specify that the user desires or prefers to have at least one hour of focus-intensive work, such that it is not required. In one embodiment, a graphical user interface for receiving user preferences may include a touch pad, buttons, windows, and/or a slider corresponding to the degree of user desire for a particular preference, further enabling the user to tune their optimal schedule(s). For example, a user may indicate a preference for having work-related meetings scheduled in the afternoon, having the scheduling align with certain other persons' schedules, or a preference for having other work-related meetings scheduled in sequence (i.e., batching the meetings back-to-back). Implicit feedback as described herein may be stored or configured via user preferences 246 as well.

By way of example and not limitation, explicit preferences or settings indicated by a user may include user preferences about specific activities (and related information) that the user desires be explicitly included, or omitted, or categories of activities to be considered or omitted, crowd-sourcing preferences, such as whether to use crowdsourced information, or whether the user's activity pattern information may be shared as crowdsourcing data; preferences about which optimal activity pattern consumers may consume the user's activity pattern information; and thresholds (such as thresholds for determining user activity patterns or evaluating confidence scores, which may be used for generating optimal schedules), and/or notification preferences, as described herein.

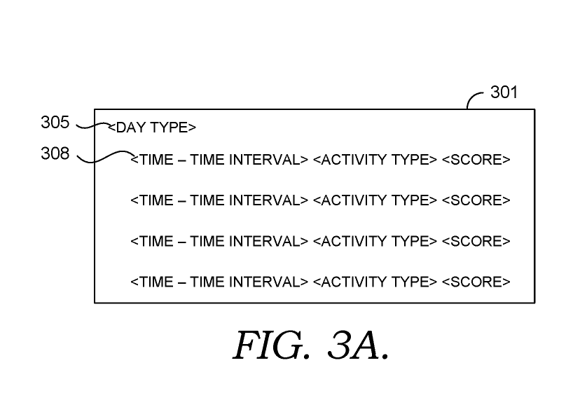

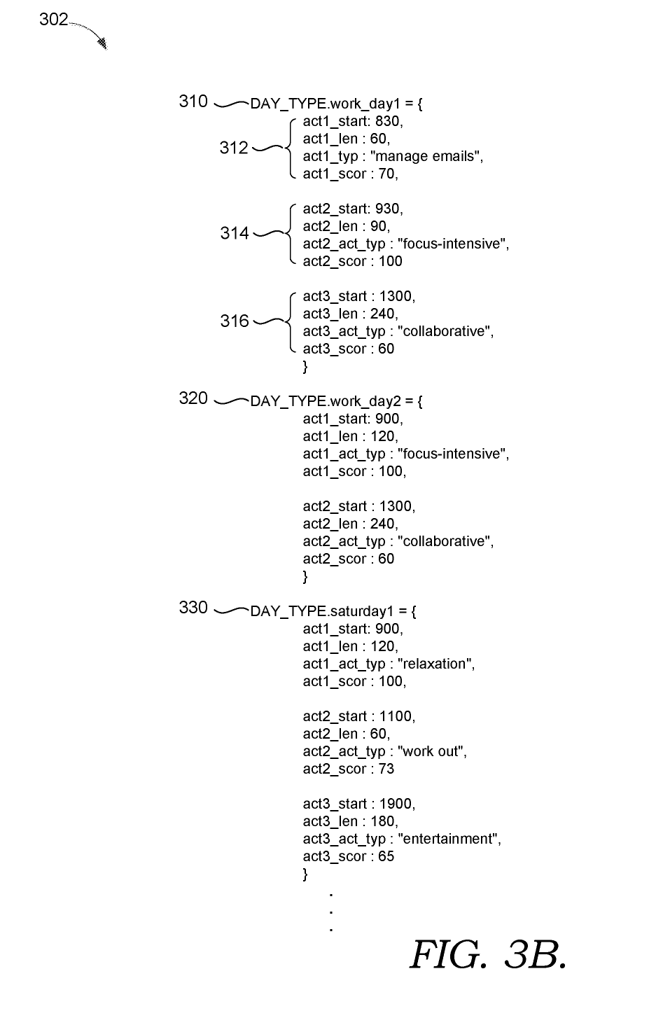

Turning now to FIGS. 3A and 3B, two example aspects of optimal schedules are illustratively depicted, as optimal schedule aspects 301 and 302, respectively. Example aspects 301 and 302 are provided merely to illustrate example features that may be included in an optimal schedule and are not intended to be limiting. The specific example formatting, structure, variable names, order, and terminology and other features of example aspects 301 and 302 are intended to show some examples of the information included in an optimal schedule and how the information may be formatted. In particular, any suitable format for an optimal schedule may be utilized by embodiments herein for us by one or more consumer applications 270 to utilize and provide an enhanced computer experience to a user. Additionally, it is contemplated that in some embodiments, optimal schedules generated by optimal schedule generator 290 may include computer-executable instructions and/or may be provided via an API to facilitate their use by consumer applications 270, which may include third-party developed computing applications or services.

Example optimal schedule aspect 301 depicts a mark-up-language-style format for an optimal schedule, which may include a number of day-type objects 305. Day-type objects 305 may include optimal schedule information for various types of days, such as workdays, weekends, odd days, even days, first day of the month, last day of the month, Mondays, Fridays, travel days, holidays, or the like. In some embodiments, day-type objects 305 may include multiple types of days, such as different type of workdays. The different types of workdays, for example, may correspond to different scenarios of the user, as determined from user activity patterns. For instance, a first work day object type may include optimal schedule information corresponding to a typical day at work, for the user. A second work day object type may correspond to a work day when the user arrives to work later than normal. As will be understood, when people arrive late to work, their schedules are typically different than a normal workday; for instance, they may stay later or skip lunch. A third workday object type may correspond to a workday when a user has a major project due in the near future. (For example, historic user activity used to generate user activity patterns for this scenario may indicate that a user spends more time on focus-intensive activity or collaborative activity and less time on recreational activity.) As further described herein, in some embodiments, optimal schedule generator 290 or a consumer application 270 may use current context information of a user to select a particular optimal schedule (sometimes referred to herein as a candidate optimal schedule), which may include determining an appropriate day-type object 305. For example, if a user's current context indicates the user arrived late to work (which may be determined based on user location information received form user-data collection component 210 or user activity monitor 280), then a day-type object 305 corresponding to a workday when the user arrives late may be selected over a normal workday.

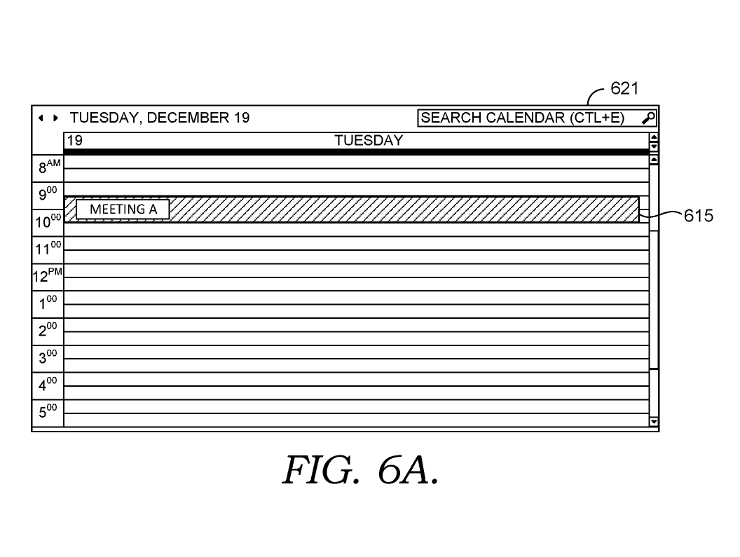

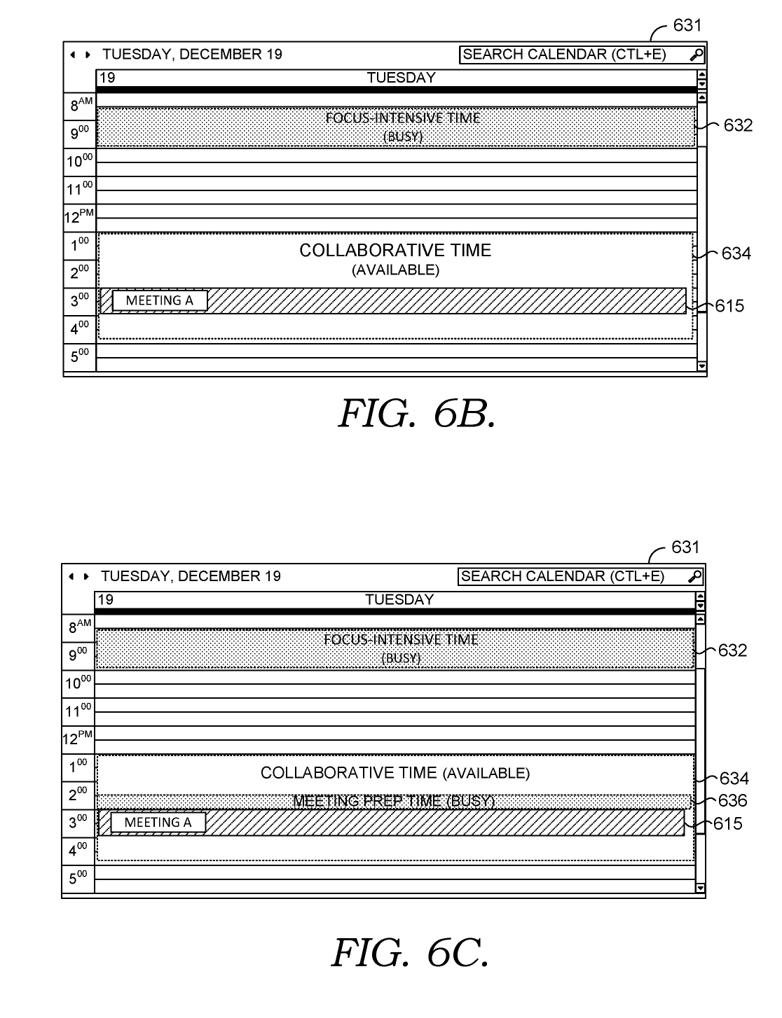

Example optimal schedule aspect 301 further includes activity object 308, which in some embodiments comprises an allocation of time corresponding to a particular future activity or activity type likely to be performed or desired to be performed by the user. As shown, example activity object 308 includes information indicating a time or time interval for the allocated future activity, the activity type (or the specific future activity), and a corresponding score (which is described in FIG. 3B). The time or time interval indicates the time or time frame for the allocated activity, may include a specific time, such as 900-1100 hours, or a start time and interval length (e.g., 900 hours, 120 minutes, respectively). In some embodiments, an optimal schedule may include conditional logic regarding times and/or activity types. For example, a user activity pattern may indicate that when certain types of meetings are scheduled for the user (such as meetings with the user's supervisor), then the user typically prepares for the meeting. Thus an optimal schedule may include conditional logic so that if a meeting is scheduled (which may be scheduled at an appropriate time consistent with the optimal schedule, such as an allocation of time designated as collaborative activity), then an allocation of time prior to the meeting is reserved for the user to prepare for the meeting. An example of this scenario is described in connection to FIGS. 6B and 6C.

Example optimal schedule aspect 302 depicts an example format in a key-value-pair-style, similar to a JSON object, which may include a number of day-type objects 310, 320, and 330, corresponding to day_type.work_day1, day_type.work_day2, and day_type.saturday1, respectively. In some embodiments, example day-type objects 310, 320, and 330 may have different types of days similar to what is described in day-type object 305 described in connection to FIG. 3A. For example, day-type object 310 and day-type object 320 comprise optimal schedule information for different types of workdays, and day-type object 330 comprises optimal schedule information for a Saturday.

As shown in example optimal schedule aspect 302, each day-type object includes one or more allocations of future activity or activity type, such as example allocations 312, 314, and 316 for day-type object 310. The allocations may correspond to intervals of allocated time throughout a day, a week, or other time frame. In this example format, each allocation of time includes a number of key-value pairs specifying information about the allocation. For example, allocation 312 includes the following key-value pairs: act1_start: 830, indicating that the allocation for activity 1 begins at time 8:30 AM; act1_len: 60 which specifies a length of time for the allocation, in minutes (here, activity 1 is allocated 60 minutes); act1_typ: “manage emails”, which specifies an activity or type of activity for the user to perform in allocation 312 (here, the activity is managing the user's emails, such as responding to emails. Allocations 314 and 316 include other examples of activity types including “focus intensive” activity and “collaborative” activity, respectively); act1_score: 70, which indicates a score corresponding to allocation 312, and which is described below. Thus, example day-type object 310 includes three allocations of time for three activities including a first activity (corresponding to allocation 312, from 8:30 to 9:30 and for managing emails), a second activity (corresponding to allocation 314, from 9:30 to 11:00 and for focus-intensive activity), and a third activity (corresponding to allocation 316, from 13:00 to 17:00 and for collaborative activity). Example day-type object 330 (corresponding to a Saturday) shows additional activity types including relaxation, workout, and entertainment.

In some embodiments, optimal schedule aspect 301 or 302 includes information indicating a score corresponding to a particular time allocation of future activity or activity type, such as the score variable shown in example activity object 308. In embodiments that include a score, the score represents a strength or degree of certainty (or likelihood) that a user will follow or desires to follow the activity type in the allocated timeframe. In particular, as described herein future activities or activity types may be determined from activity patterns of the user, which may include a confidence value or score corresponding to the strength of the pattern (which may indicate how often the user follows the pattern). In some instances, a score may be set (or determined) from user preferences. For example, where a user has explicitly configured settings to indicate that the user desires focus-intensive work for a particular time each work day, then the score may be set to 100. In some embodiments, the score may be used by a consumer application 270, such as to resolve conflicting allocations, which may arise based on a user's current context (e.g., strongest score wins) or to vary the particular service or function that is performed by the consumer application. For instance, allocation 314 has a score of 100, which may signal that a user strongly desires this allocation. Therefore notifications consumer application 273 may notify other people not to disturb the user during this time, and a calendar management consumer application 271 may indicate that the user is busy during this time or may not schedule meetings during this time.

With reference now to FIGS. 6A-6C, an illustrative example of an enhanced user experience is explained, which may be provided by a calendar managing application, such as an embodiment of consumer application 271. In this example the user receives a meeting event (i.e., a meeting invite or meeting request) for meeting A. Turning first to FIG. 6A, a calendar application 621 is depicted. Calendar application 621 depicts a conventional user calendar application, which may be used for managing a user calendar including scheduling calendar events and providing the user with a graphical depiction of their schedule. Calendar application 621 has received the meeting event for meeting A, which is shown as item 615 and scheduled from 9:30 to 10:30 AM on Tuesday, December 19. Based on information indicated in calendar application 621, the user had availability during this time.

Turning now to FIGS. 6B and 6C, an example calendar application 631 is depicted. Example calendar application 631 includes an embodiment of the technology described herein, such as calendar managing consumer application 271. In this example, calendar application 631 depicts the user's schedule for Tuesday, December 19. As shown in FIG. 6B, the user's calendar on this day is populated with calendar items 632 and 634, which may be derived from an optimal schedule generated for the user. In particular, in this example, these calendar items are based on day-type object 320 (which is a workday) of example optimal schedule aspect 302, shown in FIG. 3B. Calendar item 632 indicates focus-intensive activity from 8:30 AM to 10:00 AM, and further indicates that the user is busy during this time (and thus unavailable for a meeting event, such as meeting A). Calendar item 634 indicates collaborative time from 1:00 PM to 5:00 PM, and further indicates that the user is available. Accordingly, using example calendar application 631, meeting A has been scheduled at 3:00 PM (as shown at item 615), which is within the allocation for collaborative time and at a time interval when the user is available. Meeting A was not scheduled at 9:30 AM (as it was when using the conventional calendar application 621) because the calendar application 631 has, based on the optimal schedule, designated the time interval from 8:30 AM to 10:00 AM as focus-intensive activity and indicated the user is unavailable. Moreover, because “collaborative time” is intended to cover meetings, meeting A is scheduled for an available time frame within the collaborative activity time interval (calendar item 634). Accordingly, a user's optimal work pattern is preserved because the meeting is not scheduled during a time that has been determined to be when the user performs focus-intensive work (i.e., the morning from 8:30 AM to 10:00 AM).

Continuing with this example, FIG. 6C depicts the same calendar application 631 with calendar items 632 and 634 indicating an allocation of focus-intensive activity and collaborative activity, respectively, and showing meeting A (item 615) scheduled at 3:00 PM, during the collaborative activity time interval 634. As described above, in some embodiments, an optimal schedule may include conditional logic regarding time intervals and/or activity types, which may be used by a consumer application (such as calendar application 631 or another consumer application 270) when utilizing the optimal schedule.

Suppose that this user has a pattern of behavior of spending thirty minutes preparing for certain types of meetings, such as meeting with the user's supervisor. For this example, let meeting A be a meeting that includes the user's supervisor. This may be determined based on contextual information derived from the meeting event (i.e., the meeting request or meeting invite), which may be determined using contextual information extractor 284, and which indicates the user's current context. For example, the user has meeting with her supervisor. Accordingly, conditional logic in the optimal schedule may specify that when meeting events having certain criteria (i.e., future activities having certain activity features) are scheduled, then the user's schedule should also allocate additional time for meeting preparation. Thus calendar application 631 in FIG. 6C shows calendar item 636, which indicates meeting preparation activity from 2:30 PM to 3:00 PM, and further indicates that the user is unavailable.

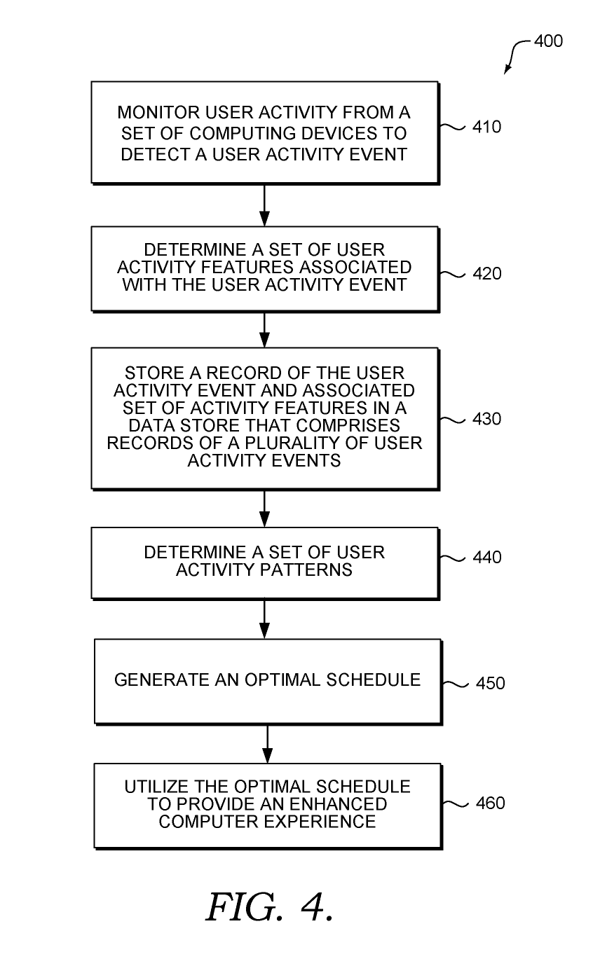

Referring now to FIG. 4, a flow diagram is provided illustrating one example method 400 for providing enhanced computer experiences using an optimal schedule. Each block or step of method 400 and other methods described herein comprises a computing process that may be performed using any combination of hardware, firmware, and/or software. For instance, various functions may be carried out by a processor executing instructions stored in memory. The methods may also be embodied as computer-useable instructions stored on computer storage media. The methods may be provided by a stand-alone application, a service, a hosted service (stand-alone or in combination with another hosted service), or a plug-in to another product, to name a few possibilities. Accordingly, method 400 may be performed by one or more computing devices, such as example computing device 700 described in FIG. 7, which may comprise a smartphone or other user device, a server, or by a distributed computing platform, such as in the cloud.

At step 410, monitor user activity from a set of computing devices to detect a user activity event. Embodiments of step 410 may include monitoring user data for user-device related information. The set of computing devices may be associated with a user, and may have sensors associated therewith that are configured to provide sensor data related to the functioning of the computing devices and activity performed by the user via the computing devices and/or applications thereof. In some instances, an activity event may comprise a series or sequence of user interactions with one or more user devices. Some embodiments of step 410 may comprise monitoring sensor data from the sensor(s) associated with the set of computing devices. Further, some embodiments of step 410 may use activity event logic to detect the user activity event, as described in connection with logic 230 of system 200 shown in FIG. 2.

In some embodiments, the devices monitored in step 410 may comprise one user device or a plurality of user devices, such as user devices 102a through 102n, described in connection to FIG. 1, monitored using one or more sensors, such as sensors 103a and 107, described in connection with FIG. 1, or other sensors which may be associated with common or separate computing devices. Such activity events may be monitored by an activity monitor, such as the user activity detector 282 of activity monitor 280 described in connection with FIG. 2.

At step 420, determine a set of user activity features associated with the user activity event. Upon detecting or otherwise identifying a user activity event in step 410, embodiments of step 420 determine a set of activity features associated with the detected activity event. Some embodiments of step 420 determine the set of activity features based at least in part on sensor data (including data that may be derived from sensor data such as interpretive data) provided by the one or more sensors associated with the set of computing devices. In some embodiments, the sensor data may be provided via a user data collection component as described in connection with FIG. 2. In particular, user data related to the detected activity event, which may be determined at least in part from sensor data, and which may include interpretive data, contextual data, and/or semantic information related to the detected activity event, is received and analyzed to determine a set of one or more features associated with the user activity.

As described previously in connection with user activity monitor 280 and activity features determiner 286 of FIG. 2, activity-related features (or variables) associated with the user activity may be used for identifying patterns of user activity. The activity features may be determined from information about a user activity, which in some embodiments may include related contextual information about the detected activity. Additional details of determining contextual information related to a detected activity event are described below and in connection to contextual information extractor 284 of FIG. 2. Further, in some embodiments, the contextual information may include semantic information determined from a semantic analysis performed on the detected activity event and/or one or more activity features associated with the activity event. For example, while a user-activity feature may indicate a specific website visited by the user, semantic analysis may determine the category of the website, related websites, themes or topics or other entities associated with the website or user activity. From the semantic analysis, additional user activity-related features semantically related to the user activity may be determined and used for identifying user activity patterns. Additional details of determining activity features that include semantic information related to the detected activity event are described in connection to semantic information analyzer 262 of FIG. 2.

Examples of activity-related features may include, without limitation, location-related features, such as location of the user device(s) during the user activity, prior to and/or after the user activity, venue-related information associated with the location, or other location-related information; time related features, such as time(s) of day(s), day of week or month of the user activity, or the duration of the activity, or related duration information such as how long the user used an application associated with the activity; content-related features, such as online activity by the user (e.g., searches, browsed websites, type or category of websites, purchases, social networking activity, communications sent or received including social media posts, which may comprise online activity occurring prior to or after the detected activity event); other features that may be detected concurrent with the user activity or near the time of the user activity; or any other features that may be detected or sensed and used for determining a pattern of the user activity including semantic features that may characterize aspects of the activity, the nature or type of the activity, and/or user interactions making up the activity.

Features may also include information about user(s) using the device; and other contextual features such as user device-related features, usage-related features; or other information about the user. By way of example and not limitation, user device-related features may include information about device type (e.g., desktop, tablet, mobile phone, fitness tracker, heart rate monitor, etc.), hardware properties or profiles, OS or firmware properties, device IDs or model numbers, network-related information (e.g., mac address, network name, IP address, domain, work group, information about other devices detected on the local network, router information, proxy or VPN information, other network connection information, or the like), position/motion/orientation related information about the user device, power information such as battery level, time of connecting/disconnecting a charger, user-access/touch information; usage-related features may include information about file(s) accessed, app usage (which may also include application data, in-app usage, concurrently running applications), network usage information, user account(s) accessed or otherwise used (such as device account(s), OS level account(s), or online/cloud-services related account(s) activity, such as with a Microsoft® account, online storage account(s), email, calendar, or social networking accounts, etc.); other information identifying a user may include login password, biometric data, which may be provided by a fitness tracker or biometric scanner; and/or characteristics of the user(s) who use the device, which may be useful for distinguishing users on devices that are shared by more than one user.

In some embodiments of step 420, user activity event logic (described in connection with user activity detector 282) may be utilized to identify specific features associated with the detected user activity event. Some implementations of step 420 may be carried out using an activity features determiner component, as described in system 200 of FIG. 2. Additional details of embodiments of step 420 are provided in connection with activity features determiner component 286, in FIG. 2.

At step 430, store a record of the user activity event and the associated set of activity features in a data store that comprises records of a plurality of user activity events. Each record in the data store may include information about a particular activity event including one or more activity features associated with the activity event. In some embodiments, the plurality of records in the activity event data store comprise records of other activity events determined according to steps 410 through 430 of method 400. In some instances, some of the other records may include information about activity events (including associated activity features) derived from other users determined to be similar to the particular user, as described previously. In an embodiment, the activity event data store comprises a user profile, and may be stored in a user activity information component, such as user activity information component 242 of user profile 240 described in connection to FIG. 2.

At step 440, determine a set of activity patterns. In embodiments of method 400, the set of activity patterns may be determined based on an analysis of the user activity events and their associated sets of activity features. In embodiments of step 440, the activity pattern may be detected based on an analysis of at least a portion of the plurality of activity event records to identify a set of activity events having similar activity features. Thus, embodiments of step 440 may comprise determining a set of user activity events that have in-common features, which may be determined using a features similarity identifier component, such as features similarity identifier 264, described in connection with system 200 of FIG. 2.

In embodiments, the set of activity events having similar activity features may comprise two or more activity events that form the basis of the pattern, as described previously in connection with activity pattern determiner 266. In some embodiments, the similar activity features may be in common with two or more of the activity events, may be substantially similar, or may be similar enough according to a similarity threshold, as described previously. Further, some embodiments may determine a corresponding confidence score associated with the activity pattern. In some instances, the confidence score may indicate a likelihood that the user will behave according to the pattern, and may be used for determining whether to provide activity pattern information to an optimal-schedule consumer application, such as those described in connection with FIG. 2. In some embodiments, one or more pattern-based predictors may be utilized, such as those described in connection with FIG. 2.

At step 450, generate an optimal schedule of future activity for the user. In some embodiments, optimal schedules of future activity may be generated to include one or more future time intervals having corresponding future activity types in accordance with the determined set of activity patterns. Further and as described herein, an optimal schedule may be based on activity likely to be performed by the user, which may be determined or inferred based on learned activity patterns and/or explicit information about a user's future activity (which may be derived from a calendar, task list, or other user data, for example). The generated schedule(s) may be considered optimal for the user by being consistent with learned behavior patterns (or activity patterns) of the user, and may be generated by an optimal schedules generator, such as the optimal schedule generator 290 described in connection with FIG. 2. Additionally, in some embodiments, current context of the user and/or user preferences or settings may be used to generate optimal schedules.

In some embodiments, multiple optimal schedules (e.g., candidate optimal schedules) may be generated, which may be mutually exclusive, allowing an optimal schedule that best matches a user's needs and/or current context to be selected. Further, a generated optimal schedule may be more likely to include an allocation of time for a user activity or activity type indicated by a strong activity pattern, which may be determined based on confidence scores as described herein in connection with FIG. 2. In some embodiments, an optimal schedule may further be determined based on user-feedback that is express or implied (e.g., express feedback such as user-indicated preferences, task lists, rules or settings, for example, or implied feedback, such as user behavior in response to recommended user activity schedules (e.g., being non-responsive), for example, and based on current context of a user, which allows an optimal schedule to be generated that aligns with a user's circumstances.

Additionally, optimal schedule logic, such as logic 235 described in connection with FIG. 2, may be used for generating the activities included in the optimal schedule, and may include rules, associations, conditions, predictions, classification models, and/or scheduling algorithms that utilize user activity, user activity patterns, predicted or future user activity, and historical user data to determine how to generate optimal user activity schedules. Additional details and embodiments for generating an optimal schedule in step 450 are described in connection to optimal schedule generator 290 in FIG. 2.

At step 460, utilize the optimal schedule to provide an enhanced computer experience. An enhanced computer experience may be provided through one or more optimal-schedule consumer applications, such as example consumer applications 271, 272, and 273 discussed in connection with FIG. 2. In accordance with FIG. 2, three example optimal-schedule consumer applications may include a calendar management program, which utilizes an optimal schedule for managing a user's calendar events to be more in accordance with the optimal schedule; a personal performance optimizer application, which utilizes an optimal schedule to provide the user with a recommended daily schedule of user activity based on future activity to be performed by the user; and a notifications service, which utilizes an optimal schedule to manage electronic notifications to the user or to other people.

In an embodiment, the calendar management program facilitates the managing of a user's calendar by receiving optimal user activity schedules, and in some embodiments, selecting one of a number of user activity schedules and modifying the user's calendar (e.g., by adding suggested user activities as calendar items) in response to acceptance of a recommended user activity and/or an optimal user activity schedule. In another embodiment, the personal performance optimizer application may suggest a recommended user activity or a schedule of recommended activities, which may be presented to the user at certain times (e.g., first thing in the morning when a user engages with a computing device) or when a user's routine has changed (e.g., it is determined the user is “out of routine” and a new optimal schedule of user activities is appropriate). In a further embodiment, the notification service manages, modifies, and/or withholds electronic communications to and/or from the user depending on the user activity (e.g., restricting notifications to and/or from the user during focus-intensive work and allowing notifications to and/or from the user during non-focus-intensive work). The notification modification may apply to the user's computing devices and applications (e.g., hiding the user's email and/or sending automated responses) and to notifications sent from other persons (e.g., restricting, modifying, preventing, and/or otherwise interrupting communications from other persons). Additional details and embodiments of step 460 are described in connection to optimized schedule consumer applications 270 and example applications 271, 272, and 273 in system 200 of FIG. 2.

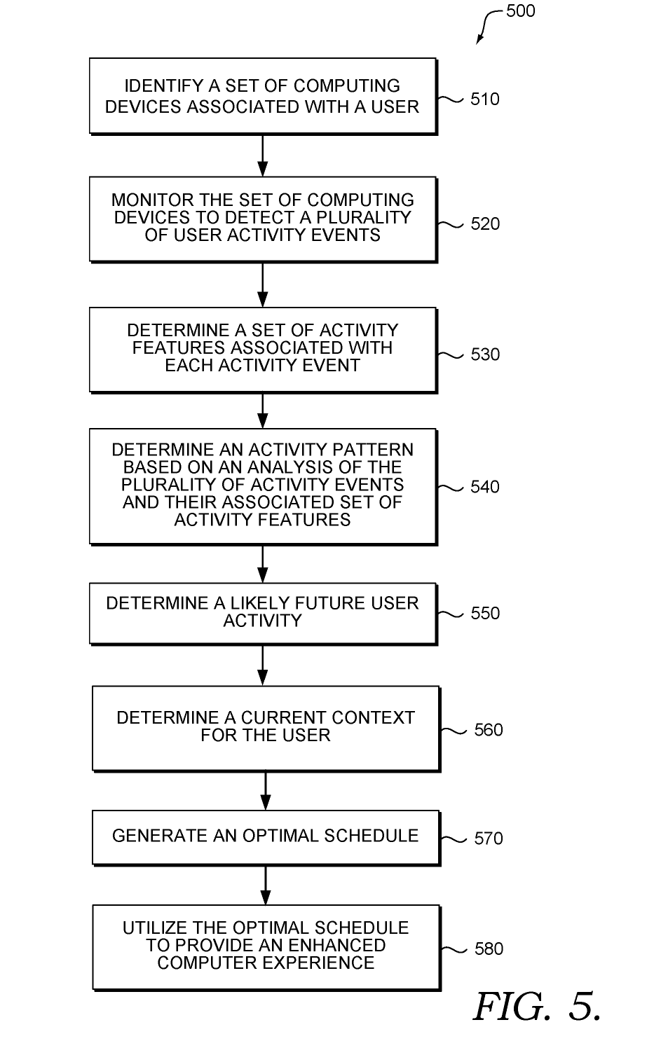

Referring to FIG. 5, a flow diagram is provided illustrating an example method 500 for providing an enhanced user experience with one or more computing devices. At step 510, identify a set of computing devices associated with a user. Embodiments of step 510 may determine a set of user devices based on monitoring user data for user device-related information. In some embodiments, the set of user devices identified in step 510 comprises one user device or a plurality of user devices, such as user devices 102a through 102n, described in connection to FIG. 1.

In one embodiment, information about user devices associated with a user may be determined from the user data made available via user-data collection component 210 described in connection to user activity monitor 280 of FIG. 2. For example, as described previously, information about a user device may be sensed or otherwise detected from user data, such as by one or more sensors associated with a user device, or may be determined by detecting and analyzing user device-related information in user data to determine characteristics of the user device, such as device hardware, software such as operating system (OS), network-related characteristics, user accounts accessed via the device, and similar characteristics. In one embodiment, the detected user devices (such as user device 102a through 102n) may be polled, interrogated, or otherwise analyzed to determine information about the devices. In some implementations, this information may be used for determining a label or identification of the device (e.g., a device ID) so that user interaction with one device may be recognized from user interaction with another device. In some embodiments of step 510, a user device may be identified based on user-provided information, such as the case where a user declares or registers the device; for example, by logging into an account via the user device, installing an application on the device, connecting to an online service that interrogates the device, or otherwise providing information about the device to an application or service.

At block 520, monitor the set of computing devices to detect a plurality of user activity events. Embodiments of step 520 may include monitoring user data for user device-related information, as discussed with respect to step 510 of method 500. The set of computing devices may be associated with a user, and may have sensors associated therewith that are configured to provide sensor data related to the functioning of the computing devices and activity performed by the user via the computing devices and/or the applications operating thereon. In some instances, an activity event may comprise a series or sequence of user interactions with one or more user devices. Some embodiments of step 520 may comprise monitoring sensor data using one or more sensors associated with the set of computing devices. Some embodiments of step 520 may use activity event logic to detect the user activity events, as described in connection with user activity detector 282. Further, some implementations of step 520 may be carried out using a user activity detector component described in system 200 of FIG. 2. Additional details of embodiments of step 520 are provided in connection with user activity detector 282 in FIG. 2 and step 410 of method 400 provided in FIG. 4.

At step 530, determine a set of activity features associated with each activity event. The activity features may be determined based at least in part on sensor data, and user data related to the detected activity event, which may be determined at least in part from sensor data and may include interpreted data, contextual data, and/or semantic information related to the detected activity event. The data may be received and analyzed to determine a set of one or more features associated with the user activity, as discussed in connection with FIGS. 2 and 4.

At step 540, determine an activity pattern based on an analysis of the plurality of user activity events and their associated set of activity features. The activity pattern may be determined as described in connection with activity pattern inference engine 260 of FIG. 2 and step 440 of FIG. 4. In an embodiment, the activity pattern may be accessed from an activity pattern data store, such as inferred activity patterns 248 of user profile 240, also described in connection with FIG. 2. As mentioned, a user may be associated with multiple activity patterns. A particular pattern can be associated with a confidence level or score that indicates how likely the pattern is to be followed.

At step 550, determine a likely future user activity. The user activity information and/or activity features associated with a user activity may be used to determine a prediction of the next action or a future action to be taken by the user. In embodiments, the determination of a likely future activity may be based on the determined activity patterns as described herein, and/or may be determined based on explicit user data such as future calendar events, task lists, user-indicated preferences, such as those stored in user preferences 246 of user profile 240, or inferred from other user data (e.g., emails, text messages, accounts information, social media account information, or other user data). Future user activity may be classified as an activity type. In some embodiments, a predicted future activity comprises a next or future user action with a highest observation count (i.e., the next user action that is predicted the most based on a set of example user actions as described with respect activity pattern determiner 266 in FIG. 2) or the highest corresponding confidence. Additional details regarding step 550 and the determination of a likely or predicted future activity are further described with respect to activity pattern inference engine 260 and optimal schedule generator 290 of FIG. 2.

At step 560, determine a current context for the user. A current context may be determined based on user data, which may include sensor data, and may provide information that relates to a user's location and circumstances (e.g., associated persons, application interaction, computing device interaction, time or day or week, indicated commitments of the user, or other types of contextual information). Further, a current context may be used for selecting between different generated candidate optimal schedules, allowing a particular optimal schedule to be selected and used by optimal-schedule consumer applications 270 that not only includes likely future user activity, but may also include user activity that is not mismatched with the current context of a user (e.g., location), as discussed in connection with FIG. 2.

At step 570, generate an optimal schedule. In one embodiment, the optimal schedule may be based on an activity pattern of the user (e.g., an inferred activity pattern based on current and/or historical user activity), or it may be based on likely future user activity, or a combination of these factors. In further embodiments, the optimal schedules of future activity are generated to include one or more future time intervals having corresponding future activity types in accordance with the determined set of activity patterns. Further, an optimal schedule may be based on activity likely to be performed by the user and also may be determined or inferred and used by the computing system in conjunction with learned activity patterns. Additionally, in some embodiments, current context of the user, user preferences, and/or settings may be considered when generating optimal schedules as discussed in connection with optimal schedule generator 290 (FIG. 2) and step 450 of method 400 (FIG. 4). Some embodiments of step 570 may be performed as described in step 450 of method 400 (FIG. 4).

At step 580, utilize the optimal schedule to provide an enhanced computer experience. An enhanced computer experience may be provided using one or more optimal-schedule consumer applications, such as example consumer applications 271, 272, and 273 described in connection with optimal-schedule consumer applications 270 and example applications 271, 272, and 273 in system 200 of FIG. 2, and step 460 of method 400 (FIG. 4).

Having described various implementations, an exemplary computing environment suitable for implementing embodiments of the disclosure is now described. With reference to FIG. 7, an exemplary computing device is provided and referred to generally as computing device 700. The computing device 700 is but one example of a suitable computing environment and is not intended to suggest any limitation as to the scope of use or functionality of embodiments of the disclosure. Neither should the computing device 700 be interpreted as having any dependency or requirement relating to any one or combination of components illustrated.

Embodiments of the disclosure may be described in the general context of computer code or machine-useable instructions, including computer-useable or computer-executable instructions, such as program modules, being executed by a computer or other machine, such as a personal data assistant, a smartphone, a tablet PC, or other handheld device. Generally, program modules, including routines, programs, objects, components, data structures, and the like, refer to code that performs particular tasks or implements particular abstract data types. Embodiments of the disclosure may be practiced in a variety of system configurations, including handheld devices, consumer electronics, general-purpose computers, or more specialty computing devices. Embodiments of the disclosure may also be practiced in distributed computing environments where tasks are performed by remote-processing devices that are linked through a communications network. In a distributed computing environment, program modules may be located in both local and remote computer storage media including memory storage devices.

With reference to FIG. 7, computing device 700 includes a bus 710 that directly or indirectly couples the following devices: memory 712, one or more processors 714, one or more presentation components 716, one or more input/output (I/O) ports 718, one or more I/O components 720, and an illustrative power supply 722. Bus 710 represents what may be one or more busses (such as an address bus, data bus, or combination thereof). Although the various blocks of FIG. 7 are shown with lines for the sake of clarity, in reality, these blocks represent logical, not necessarily actual, components. For example, one may consider a presentation component such as a display device to be an I/O component. Also, processors have memory. The inventors hereof recognize that such is the nature of the art and reiterate that the diagram of FIG. 7 is merely illustrative of an exemplary computing device that can be used in connection with one or more embodiments of the present disclosure. Distinction is not made between such categories as “workstation,” “server,” “laptop,” or “handheld device,” as all are contemplated within the scope of FIG. 7 and with reference to “computing device.”

Computing device 700 typically includes a variety of computer-readable media. Computer-readable media can be any available media that can be accessed by computing device 700 and includes both volatile and nonvolatile, removable and non-removable media. By way of example, and not limitation, computer-readable media may comprise computer storage media and communication media. Computer storage media includes both volatile and nonvolatile, removable and non-removable media implemented in any method or technology for storage of information such as computer-readable instructions, data structures, program modules, or other data. Computer storage media includes, but is not limited to, RAM, ROM, EEPROM, flash memory or other memory technology, CD-ROM, digital versatile disks (DVDs) or other optical disk storage, magnetic cassettes, magnetic tape, magnetic disk storage or other magnetic storage devices, or any other medium which can be used to store the desired information and which can be accessed by computing device 700. Computer storage media does not comprise signals per se. Communication media typically embodies computer-readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media, such as a wired network or direct-wired connection, and wireless media, such as acoustic, RF, infrared, and other wireless media. Combinations of any of the above should also be included within the scope of computer-readable media.

Memory 712 includes computer storage media in the form of volatile and/or nonvolatile memory. The memory may be removable, non-removable, or a combination thereof. Exemplary hardware devices include for example solid-state memory, hard drives, and optical-disc drives. Computing device 700 includes one or more processors 714 that read data from various entities such as memory 712 or I/O components 720. Presentation component(s) 716 presents data indications to a user or other device. Exemplary presentation components include a display device, speaker, printing component, vibrating component, and the like.

The I/O ports 718 allow computing device 700 to be logically coupled to other devices, including I/O components 720, some of which may be built in. Illustrative components include a microphone, joystick, game pad, satellite dish, scanner, printer, or a wireless device. The I/O components 720 may provide a natural user interface (NUI) that processes air gestures, voice, or other physiological inputs generated by a user. In some instances, inputs may be transmitted to an appropriate network element for further processing. An NUI may implement any combination of speech recognition, touch and stylus recognition, facial recognition, biometric recognition, gesture recognition both on screen and adjacent to the screen, air gestures, head and eye tracking, and touch recognition associated with displays on the computing device 700. The computing device 700 may be equipped with depth cameras, such as stereoscopic camera systems, infrared camera systems, RGB camera systems, and combinations of these, for gesture detection and recognition. Additionally, the computing device 700 may be equipped with accelerometers or gyroscopes that enable detection of motion. The output of the accelerometers or gyroscopes may be provided to the display of the computing device 700 to render immersive augmented reality or virtual reality.

Some embodiments of computing device 700 may include one or more radio(s) 724 (or similar wireless communication components). The radio transmits and receives radio or wireless communications. The computing device 700 may be a wireless terminal adapted to receive communications and media over various wireless networks. Computing device 700 may communicate via wireless protocols, such as code division multiple access (“CDMA”), global system for mobiles (“GSM”), or time division multiple access (“TDMA”), as well as others, to communicate with other devices. The radio communications may be a short-range connection, a long-range connection, or a combination of both a short-range and a long-range wireless telecommunications connection. When we refer to “short” and “long” types of connections, we do not mean to refer to the spatial relation between two devices. Instead, we are generally referring to short range and long range as different categories, or types, of connections (i.e., a primary connection and a secondary connection). A short-range connection may include, by way of example and not limitation, a Wi-Fi® connection to a device (e.g., mobile hotspot) that provides access to a wireless communications network, such as a WLAN connection using the 802.11 protocol; a Bluetooth connection to another computing device is a second example of a short-range connection, or a near-field communication connection. A long-range connection may include a connection using, by way of example and not limitation, one or more of CDMA, GPRS, GSM, TDMA, and 802.16 protocols.

Many different arrangements of the various components depicted, as well as components not shown, are possible without departing from the scope of the claims below. Embodiments of the disclosure have been described with the intent to be illustrative rather than restrictive. Alternative embodiments will become apparent to readers of this disclosure after and because of reading it. Alternative means of implementing the aforementioned can be completed without departing from the scope of the claims below. Certain features and sub-combinations are of utility and may be employed without reference to other features and sub-combinations and are contemplated within the scope of the claims.

What is claimed is:

1. A computerized system, comprising:
one or more sensors configured to provide sensor data;
one or more processors; and
computer storage memory having computer-executable instructions stored thereon which, when executed by the one or more processors, execute a method comprising:
monitoring user activity from a set of user devices associated with a user;
determining, based on the monitored user activity, a set of activity features associated with the user activity, the set of activity features determined based at least in part on the sensor data;
storing a record of the user activity and the associated set of activity features in a data store that comprises records of a plurality of user activities;
determining a set of activity patterns based on an analysis of the user activity and the associated set of activity features;
determining, based at least on the set of activity patterns, an optimal schedule of future activity for the user; and
utilizing the optimal schedule of future activity to provide an enhanced computer experience to the user.

2. The computerized system of claim 1, wherein the optimal schedule of future activity includes one or more future time intervals having corresponding future activity types in accordance with the determined set of activity patterns.

3. The computerized system of claim 1, wherein the enhanced computer experience comprises at least one of:
a calendar-management computer application configured to manage a user's schedule in accordance with the determined set of activity patterns;
a personal performance optimizer computer application configured to generate and provide a recommendation comprising a scheduled future activity; and
a notifications application configured to manage electronic notifications associated with the user based on the determined set of activity patterns.

4. The computerized system of claim 3, wherein the notifications application selectively withholds or modifies electronic notifications based on the determined optimal schedule of future activity.

5. The computerized system of claim 3, wherein managing a user's schedule in accordance with the determined set of activity patterns by the calendar-management computer application includes:
determining a future user activity;
classifying the future user activity as an activity type; and
automatically scheduling the future user activity on a calendar associated with the user based on the activity type and the optimal schedule of future activity.

6. The computerized system of claim 1, further comprising a graphical user interface configured to receive user preferences from the user, wherein the optimal schedule of future activity is further determined based on the received user preferences.

7. The computerized system of claim 1, further comprising determining, based on the monitored user activity, a current context associated with the user, wherein the optimal schedule of future activity is further determined based on the current context.

8. The computerized system of claim 7, wherein determining the optimal schedule of future activity based on the current context comprises:
determining a plurality of candidate optimal schedules;
selecting a first candidate optimal schedule based on the current context; and
providing the first candidate optimal schedule as the optimal schedule of future activity.

9. The computerized system of claim 7, wherein the current context comprises at least one of:
a current location of the user;
a current time;
a current calendar event associated with the user;
a current application engaged with by the user; and
a current content engaged with by the user.

10. The computerized system of claim 1, wherein the optimal schedule of future activities further comprises designations for a presence indicator associated with the user for each of the future activities.

11. A computer software product configured to operate on one or more computing devices and provide an enhanced computer experience to a user, the computer software product comprising:
a monitoring component configured to monitor user activity from a set of computing devices associated with the user, at least a subset of the set of computing devices having associated therewith one or more sensors configured to provide sensor data;
a user interface; and
computer-executable instructions that, when executed by at least one processor of the one or more computing devices, perform a method comprising:
determining, based on monitored user activity from the monitoring component, a set of activity features associated with the user activity, the set of activity features determined based at least in part on the sensor data;
storing a record of the user activity and associated set of activity features in a data store that comprises records of a plurality of user activities;
determining a set of activity patterns based on an analysis of the user activity and the associated set of activity features;
determining, based at least on the set of activity patterns, an optimal schedule of future activity for the user; and
utilizing the optimal schedule of future activity to provide an enhanced computer experience to the user.

12. The computer software product of claim 11, wherein the optimal schedule of future activity includes one or more future time intervals having corresponding future activity types in accordance with the determined set of user activity patterns.

13. The computer software product of claim 11, wherein the enhanced computer experience comprises at least one of:
a calendar-management computer application configured to manage a user's schedule in accordance with the determined set of activity patterns;
a personal performance optimizer computer application configured to generate and provide a recommendation comprising a scheduled future activity; and
a notifications application configured to manage electronic notifications associated with the user based on the determined set of activity patterns.

14. The computer software product of claim 13, wherein the notifications application selectively withholds or modifies electronic notifications based on the determined optimal schedule of future activity.

15. The computer software product of claim 13, wherein the method further comprises managing a user's schedule in accordance with the determined set of activity patterns using the calendar-management computer application by:
determining a future user activity;
classifying the future user activity as an activity type; and
automatically scheduling the future user activity on a calendar associated with the user based on the activity type and the optimal schedule of future activity.

16. The computer software product of claim 11, wherein the user interface is configured to receive user preferences from the user, and wherein the optimal schedule of future activity is further determined based on the received user preferences.

17. The computer software product of claim 11, wherein the method further comprises determining, based on the monitored user activity, a current context associated with the user, wherein the optimal schedule of future activity is further determined based on the current context.

18. A computerized method for providing an enhanced user experience with one or more computing devices, the method comprising:
identifying a set of computing devices associated with a user, at least a subset of the set of computing devices having associated therewith one or more sensors configured to provide sensor data;
monitoring the set of computing devices to detect a plurality of user activities;
determining, based on the plurality of user activities, a set of activity features associated with the plurality of user activities, the set of activity features determined based at least in part on sensor data from at least a first computing device from the subset of the set of computing devices;
determining an activity pattern based on an analysis of the plurality of user activities and the associated set of activity features;
predicting a probable future user activity based on the determined activity pattern;
determining a current context based at least in part on the sensor data;
determining an optimal schedule for the user based at least on the probable future user activity; and
utilizing the optimal schedule to provide an enhanced computer experience to the user.

19. The computerized method of claim 18, wherein the optimal schedule includes one or more future time intervals having corresponding future activity types selected based on the determined activity pattern.

20. The computerized method of claim 18, wherein the enhanced computer experience comprises at least one of:
a calendar-management computer application configured to manage a user's schedule in accordance with the determined activity pattern;
a personal performance optimizer computer application configured to generate and provide a recommendation comprising a scheduled future activity; and
a notifications application configured to manage electronic notifications associated with the user based on the determined activity pattern.
